(19) United States
(12) Patent Application Publication
(10) Pub. No.: US 2008/0058383 A1
(43) Pub. Date: Mar. 6, 2008

(54) Androgen Receptor Modulators

(76) Inventors: Henrik Jernstedt, Uppsala (SE); Neeraj Garg, Tumba (SE); Annika Gustavsson, Ekero (SE); Mikael Gillner, Stockholm (SE); Ana Maria Garcia Collazo, Stockholm (SE); Eva Koch, Stockholm (SE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/576,777

(22) PCT Filed: Oct. 21, 2004
(86) PCT No.: PCT/GB04/04464

§ 371 Date: Jun. 12, 2007

(30) Foreign Application Priority Data
Oct. 21, 2003 (EP) 0324551.1

(51) Int. Cl. A61K 31/44; A61P 5/28; C07D 211/00; C07D 213/02; C07D 315/00

(52) U.S. Cl. 514/336; 514/345; 514/349; 546/290; 546/297; 549/416; 549/424

(57) ABSTRACT

Treatment of Diseases caused by Disturbances of the Activity of the Androgen Receptor uses of compounds of Formula (I): (as defined herein), for the treatment of diseases caused by disturbances of the activity of androgen receptor are provided: Formula (I). Isolated compounds of Formula (I) are also provided.

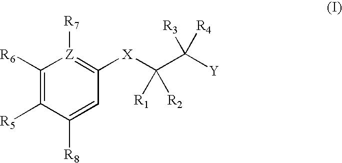

### CROSS-REFERENCE TO RELATED APPLICATIONS

This is an application filed under 35 USC §371 of International Application No. PCT/GB2004/004464 filed 21 Oct. 2004.

### FIELD OF INVENTION

This invention relates to novel compounds which are androgen receptorligands, to methods of preparing such compounds and to methods for using such compounds such as for androgen hormone replacement therapy and for diseases modulated by the androgen receptor such as benign prostatic hyperplasia, prostate cancer, alopecia, hirsutism, bone loss, bone fractures, osteoporosis, cachexia, and muscle wasting.

### BACKGROUND OF INVENTION

The androgen receptor (AR) is a member of the steroid hormone nuclear receptor family of ligand activated transcription factors. This group includes estrogen, progesterone, mineralocorticoid, and glucocorticoid receptors all of which are activated by endogenous steroid hormones to control the expression of responsive genes. The hormone receptors share a modular structure consisting of a variable amino-terminal domain (NTD), a highly conserved DNA-binding domain (DBD), and a carboxy-terminal ligand-binding domain (LBD). The DNA-binding domain generates much of the transcriptional specificity due to its ability to discern different DNA response elements with the promoter regions of target genes. The LBD is required for ligand dependent transcriptional activity containing both the hormone-binding pocket and an important transcriptional activation functional region (AF2) required for recruitment of coactivators and the cellular transcriptional machinery.

Regulation of nuclear receptor activity resides predominantly in the binding of the hormone ligand within the LBD. The amino acids lining the interior of the hormone-binding cavity define the selectivity of the receptor for its hormone. This allows AR to discriminate between the natural ligands and non-natural ligands.

Another level of transcriptional control is conveyed by the nuclear receptor's environment. It is widely accepted that different effector proteins (coactivators and corepressors) exist within different cell types and can lead to different patterns of gene expression. Because the conformational state of the receptor dictates which coactivator is recruited in a given cell type, it also imparts transcriptional selectivity. It is precisely this type of control that gave rise to tissue selective receptor modulators. For example, tamoxifen is a prototypical estrogen receptor selective modulator with differing properties within breast and uterine tissues. Exploitation of the conformational changes induced by synthetic ligands within the hormone-binding cavity has lead to multiple generations of tissue selective receptor modulators for the estrogen receptor and can be applied to developing modulators of other nuclear receptors such as the androgen receptor.

The use of natural and synthetic androgen in hormone replacement therapy has been shown to markedly decrease the risk of osteoporosis and muscle wasting. In addition, there is evidence that hormone replacement therapy has cardiovascular benefits. However hormone replacement therapy is also associated with an increase risk of prostate cancer. It is known that certain types of synthetic AR ligands display a mixed agonist/antagonist profile of activity showing agonist activity in some tissues and antagonist activity in other tissues. Such ligands are referred to as selective androgen receptor modulators (SARMS).

What is needed in the art are compounds that can produce the same positive responses as androgen replacement therapy without the negative side effects. Also needed are androgen-like compounds that exert selective effects on different tissues of the body.

The amino acids and the “space” they define as the hormone-binding cavity can be exploited in synthesizing modulators that are high receptor selective. These interactions between the endogenous hormone and amino acid residues within the ligand-binding cavity induce conformational changes that are distributed throughout the entire receptor structure. It is these conformational changes that lead to the dissociation of chaperone proteins that stabilize the receptors in the absence of ligand and the association of coactivator proteins. A liganded receptor devoid of its chaperone proteins is able to dimerize, translocate, recruit coactivators, and initiate transcription.

The natural ligand for the androgen receptor, androgen, is produced in both men and women by the gonads, adrenal glands and locally in target tissues. The levels of androgens secreted by the gonads are tightly regulated by a feedback mechanism involving the hypothalamus and pituitary.

In men, androgens are necessary for masculinization and fertility. However, systemic androgen excess causes testicular atrophy and infertility. Androgens may also contribute to lipid abnormalities, cardiovascular disease and psychological abnormalities. Local androgen excess is implicated in the pathogenesis of male pattern baldness (alopecia), benign prostatic hyperplasia (BPH) and acne. The physiologic role of androgens in women is not well understood, but these steroids do play a role in the development of normal body hair and libido. In women, relative androgen excess causes hirsutism (excessive hair growth), amenorrhea (abnormal loss or suppression of menses), acne and male pattern baldness.

The risk of developing prostate cancer increases dramatically with age. More than 75% of prostate cancer diagnoses are in men over the age of 65, and the prevalence of clinically undetectable prostate cancer in men over 80 years old is as high as 80%. It remains unclear as to the exact cause of prostate cancer, however, it is widely accepted that androgens can increase the severity and the rate of progression of the disease. Androgen deprivation therapy has been the basis for prostate cancer therapy since 1941 when castration was shown to have beneficial effects on advanced stages of the disease. Hormonal intervention is currently based on disrupting the hypothalamus-pituitary-gonadal feedback mechanism to control the levels of endogenous androgens from the testes. Antiandrogens are incorporated in later stage therapies to work at the level of the androgen receptor itself, blocking residual androgens from adrenal sources. In spite of these treatments, there exists a need for an improved therapy of diseases linked to disturbances in the activity of the androgen receptor.

### SUMMARY OF THE INVENTION

The present invention provides the use of a compound according to Formula I for the preparation of a medicament, wherein Formula I is defined as:

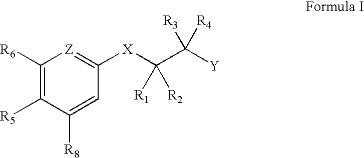

in which;

R₁ and R₂ are the same or different and independently selected from hydrogen, halogen, C₁-C₁₀ alkyl, C₁-C₁₀ substituted alkyl, C₂-C₁₀ alkenyl, C₂-C₁₀ alkynyl, C₁-C₁₀ alkoxy, C₁-C₁₀ alkenoxy, C₁-C₁₀ alkynoxy, C₁-C₁₀ alkylthio, C₁-C₁₀ alkenylthio, C₁-C₁₀ alkynylthio, C₆-C₁₀ arylthio, C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alknylsulphoxide, C₆- C₁₀ arylsulphoxide, C₁-C₁₀ alkylarylthio, C₁-C₁₀ alkylarylsulphone, C₁-C₁₀ alkylarylsulphoxide, C₆-C₁₀ aryl, or C₅-C₂₀ heteroaryl, optionally substituted with 0, 1, 2 or 3 groups of R^{a} which groups may be the same or different; or R₁ and R₂ may together form a C₃-C₁₀ cycloalkyl group;

R₃ and R₄ are the same or different and independently selected from hydrogen, halogen, C₁-C₂₀ alkyl, C₃-C₇ cycloalkyl, C₂-C₄ alkenyl, C₂-C₄ alkynyl, C₁-C₄ alkoxy, C₁-C₄ alkenoxy, C₁-C₄ alkynoxy, C₁-C₄ alkylthio, C₁-C₄ alkenylthio, C₁-C₄ alkynylthio C₁-C₁₀ alkylsulphone, C₁-C₁₀ alkenylsulphone, C₁-C₁₀ alkynylsulphone, C₆-Cl₀ arylsulphone, C₁-C₁₀ alkylsulphoxide, C₁-C₁₀ alkenylsulphoxide, C₁-C₁₀ alkynylsulphoxide, C₆-C₁₀ arylsulphoxide, C₁-C₁₀ alkylarylthio, C₁-C₁₀ alkylarylsulphone, C₁-C₁₀ alkylarylsulphoxide, C₆-C₁₅ aryl, C₅-C₂₀ heteroaryl optionally substituted with 0, 1, 2 or 3 groups of R^{a} which groups may be the same or different; or can together form a keto group;

R₅ is chosen from nitro, cyano, —CH₂CN, —COMe, or —SO₂CH₃;

R₆ is chosen from the group consisting of; hydrogen, C₁-C₅ alkyl, halogen, CN, CO₂H, CHF₂, CH₂F or CF₃;

Z is chosen from CR₇ or N;

R₇ is chosen from the group consisting of; H or C₁-C₅ alkyl;

R₈ is chosen from the group consisting of; hydrogen, C₁-C₅ alkyl, halogen, CHF₂, CH₂F or CF₃;

X is —NH—

Y is hydroxy or —NH(C₁-C₁₀ heteroaryl);

R^{a} represents a member selected from: hydrogen, halogen, —CN, OH, CO₂H, CHO, NO₂, —NH₂, —NH(C₁-C₄); N(C₁-C₄)₂, —NH(C₆ aryl), —N(C₆ aryl)₂, —NH(C₅-C₁₀ heteroaryl), and —N(C₅-C₁₀ heteroaryl)₂; or a pharmaceutically acceptable salt thereof.

A preferred compound is according to formula I, wherein R₁ or/and R₂ are H, (S)-methyl, methyl, (R)-ethyl, (S)-ethyl, ethyl, (R)-propyl, (S)-propyl, propyl, (S)-butyl, (S)-1-methyl-propyl, (S)-2-methyl-propyl, (R)-isopropyl, (S)-isopropyl, isopropyl, cyclopentyl, —(CH₂)₂SMe, (R)-CH₂SCH₂Ph, (S)-benzyl, 4-chloro-benzyl, (S)-3-methyl-1-H-indole or (S)-phenyl;

Further preferred is a compound according to formula I, wherein R₃ is chosen from the group consisting of; hydrogen, methyl, ethyl, phenyl, 3-hydroxy phenyl, 4-hydroxy phenyl, or forms a keto group together with R₄.

Further preferred is a compound according to formula I, wherein R₄ is H, methyl, or forms a keto group together with R₃.

Further preferred is a compound according to formula I, wherein R₅ is NO₂, CN, or CH₂CN;

Further preferred is a compound according to formula I, wherein R₆ is Me, or CF₃;

Further preferred is a compound according to formula I, wherein R₇ is H or Me;

Further preferred is a compound according to formula I, wherein R₈ is H or methyl;

Further preferred is a compound according to formula I, wherein Y is —OH;

Even more preferred is a compound according to formula I, chosen from the group consisting of:
2-Methyl-2-(4-nitro-3-trifluoromethyl-phenylamino)-propan-1-ol;
[1-(4-Nitro-3-trifluoromethyl-phenylamino)-cyclopentyl]-methanol;
(S)-2-(4-Nitro-3-trifluoromethyl-phenylamino)-3-phenyl-propan-1-ol;
(S)-2-(4-Nitro-3-trifluoromethyl-phenylamino)-butan-1-ol;
2-Methyl-2-(3-hydroxy-4-nitro-phenylamino)-propan-1-ol;
[1-(3-Methyl-4-nitro-phenylamino)-cyclopentyl]-methanol;
(S)-2-(3-Methyl-4-nitro-phenylamino)-butan-1-ol;
[1-(6-Methyl-5-nitro-pyridin-2-ylamino)-cyclopentyl]-methanol;
(S)-2-(6-Methyl-5-nitro-pyridin-2ylamino) 2-phenyl-ethanol;
(S)-2-(6-Methyl-5-nitro-pyridin-2-ylamino)-3-phenyl-propan-1-ol;
(S)-2-(6-Methyl-5-nitro-pyridin-2-ylamino)-butan-1-ol;
(DL)-3-(4-Chloro-phenyl)-2-(6-methyl-5-nitro-pyridin-2-ylamino)- -propan-1-ol;
(S)-2-(6-Methyl-5-nitro-2-pyridin-2-ylamino)-propionic acid;
(S)-2-(6-Methyl-5-nitro-pyridin-2-ylamino)-propan-1-ol;
2-(2,3-Dimethyl-4-nitro-phenylamino)-2-methyl-propan-1-ol;
(S)-2-(3,5-Dimethyl-4-nitro-phenylamino)-butan-1-ol;
4-(2-Hydroxy-1,1-dimethyl-ethylamino)-2-trifluoromethyl-benzonitrile;
4-(1-Hydroxymethyl-cyclopentylamino)-2-trifluoromethyl-benzonitrile;
(S)-4-(1-Hydroxymethyl-cyclopentylamino)-2-trifluoromethyl-benzonitrile;
(R)-4-(1-Hydroxymethyl-butylamino)-2-trifluoromethyl-benzonitrile;
(S)-4-(1-Hydroxymethyl-butylamino)-2-trifluoromethyl-benzonitrile;
[4-((S)-1-Hydroxymethyl-butylamino)-2-trifluoromethyl-phenyl]-acetonitrile;
[4-((R)-1-Hydroxymethyl-butylamino)-2-trifluoromethyl-phenyl]-acetonitrile;
[4-((S)-1-Hydroxymethyl-3-methyl-butylamino)-2-trifluoromethyl-phenyl]-acetonitrile;
4-(2-Hydroxy-1,1-dimethyl-ethylamino)-2-methyl-benzonitrile;
6-(2-Hydroxy-1,1-dimethyl-ethylamino)-2-methyl-nicotinonitrile;
4-(2-Hydroxy-1,1-dimethyl-ethylamino)-2,3-dimethyl-benzonitrile;
   and the compounds showed in the following table (The substituents, R9, R6, and Z, are shown in the table, and are all substituents in the following formula II. In formula II, the NO₂ group corresponds to the substituent R5 in formula I, and R9 is composed of the moieties XR₁R₂YR₃R₄ of Formula I as defined above.

| | |
|---|---|
| | Formula II |
| 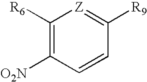 | |

| R9 | R6 | Z |
|---|---|---|
| 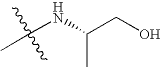 | CF₃ | CH |
| 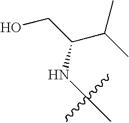 | CF₃ | CH |
| 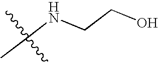 | CF₃ | CH |
| 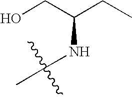 | CF₃ | CH |
| 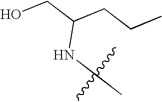 | CF₃ | CH |
| 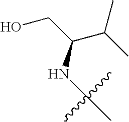 | CF₃ | CH |
| 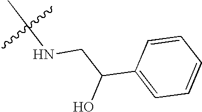 | CF₃ | CH |
| 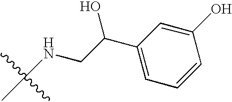 | CF₃ | CH |
| 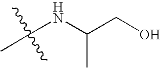 | CF₃ | CH |
| 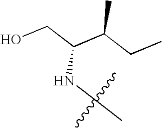 | CF₃ | CH |
| 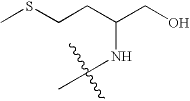 | CF₃ | CH |
| 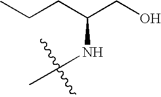 | CF₃ | CH |
| 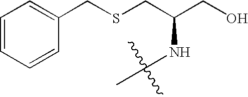 | CF₃ | CH |
| 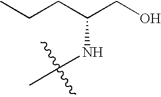 | CF₃ | CH |
| 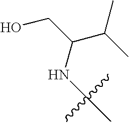 | CF₃ | CH |
| 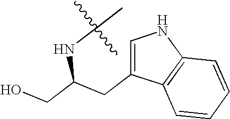 | CF₃ | CH |
| 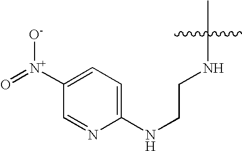 | CF₃ | CH |
| 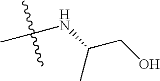 | CH₃ | N |
| 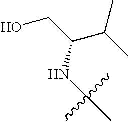 | CH₃ | N |
| 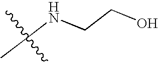 | CH₃ | N |
| 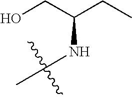 | CH₃ | N |
| 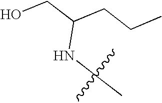 | CH₃ | N |
| 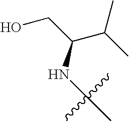 | CH₃ | N |
| 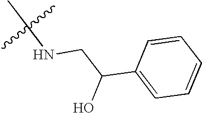 | CH₃ | N |
| 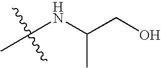 | CH₃ | N |
| 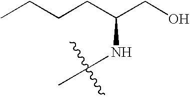 | CH₃ | N |
| 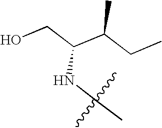 | CH₃ | N |
| 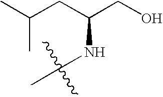 | CH₃ | N |
| 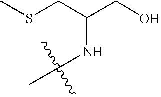 | CH₃ | N |
| 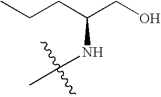 | CH₃ | N |
| 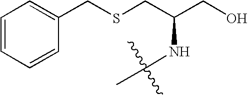 | CH₃ | N |
| 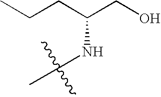 | CH₃ | N |
| 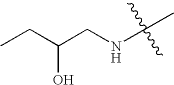 | CH₃ | N |
| 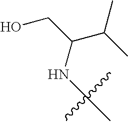 | CH₃ | N |
| 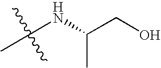 | CH₃ | CH |
| 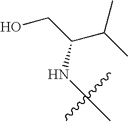 | CH₃ | CH |
| 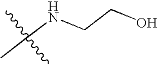 | CH₃ | CH |
| 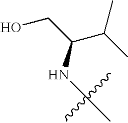 | CH₃ | CH |
| 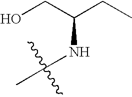 | CH₃ | CH |
| 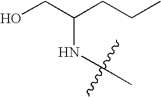 | CH₃ | CH |
| 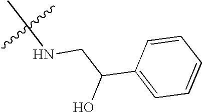 | CH₃ | CH |
| 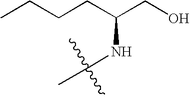 | CH₃ | CH |
| 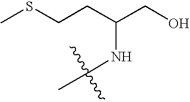 | CH₃ | CH |
| 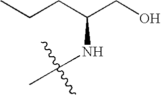 | CH₃ | CH |
| 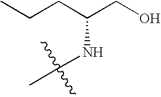 | CH₃ | CH |
| 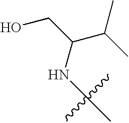 | CH₃ | CH |
| 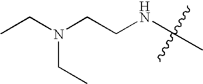 | CH₃ | CH |
| 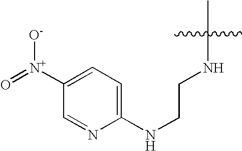 | CH₃ | CH |

2-Methyl-N-(6-methyl-5-nitro-pyridin-2-yl amino)-propan-2-ol;
4-((R)-2-Hydroxy-1-methyl-ethylamino)-2-trifluoromethyl-benzonitrile
4-((R)-1-Furan-2-ylmethyl-2-hydroxy-ethylamino)-2-trifluoromethyl-benzonitrile
(R)-3-Furan-2-yl-2-(6-methyl-5-nitro-pyridin-2-ylamino)-propan-1-ol
2-(6-Methyl-5-nitro-pyridin-2-ylamino)-heptan-1-ol
3-Cyclopentyl-2-(6-methyl-5-nitro-pyridin-2-ylamino)-propan-1-ol
[1-(4-Methanesulfonyl-3-methyl-phenylamino)-cyclopentyl]-methanol
2,2-Dimethyl-3-(6-methyl-5-nitro-pyridin-2-ylamino)-propan-1-ol
2,2-Dimethyl-3-(3-methyl-4-nitro-phenylamino)-propan-1-ol
4-((R)-1-Benzylsulfanylmethyl-2-hydroxy-ethylamino)-2-trifluoromethyl-benzonitrile
(R)-2-(6-Methyl-5-nitro-pyridin-2-ylamino)-3-phenylmethanesulfinyl-propan-1-ol
4-((R)-2-Hydroxy-1-phenylmethanesulfinylmethyl-ethylamino)-2-trifluoromethyl-benzonitrile
[1-(4-Nitro-phenylamino)-cyclopentyl]-methanol
(S)-2-(4-Nitro-phenylamino)-pentan-1-ol
[1-(2-Bromo-4-nitro-phenylamino)-cyclopentyl]-methanol
(S)-2-(2-Bromo-4-nitro-phenylamino)-pentan-1-ol
(S)-2-(2-Bromo-4-nitro-phenylamino)-4-methyl-pentan-1-ol
   or a pharmaceutically acceptable salt thereof.

Also preferred is a compound according to Formula I, wherein R₁ or R₂ is a C₆-C₁₀ arylthio moiety comprising an aryl-substituted sulfur-containing C₁-C₁₀ alkyl group.

Further preferred is a compound according to Formula I, wherein in R₁ or R₂ the alkylsulfur is substituted with a C₆ aryl group.

The present invention further provides a pharmaceutical composition which contains one or more of the compounds according to the above.

More preferred is a pharmaceutical composition according to the above, for use as a medicament.

Furthermore, the invention provides the use of a pharmaceutical composition according to the above for manufacturing a medicament to be used in the treatment of a disease caused by a disturbance in the activity of the androgen receptor.

Since the compounds are shown to be mainly antagonists for the androgen receptor, a preferred use is the use of the composition above for treating a disease which is caused by an increase in androgen receptor activity.

Even more preferred is the use of the composition above for treating a disease which is chosen from the group consisting of; prostate cancer, lipid abnormalities, cardiovascular disease and psychological abnormalities, male pattern baldness (alopecia), benign prostatic hyperplasia (BPH) and acne, hirsutism, amenorrhea, hypogonadism, anemia, diabetes, defects in spermatogenesis, cachexia, osteoporosis, osteopenia, and muscle wasting.

The present invention also provides the use of a compound according to the above for manufacturing a medicament to be used in the treatment of a disease caused by a disturbance in the activity of the androgen receptor.

A specific disease that would be amenable for treatment by the present invention is a disease chosen from the group consisting of; prostate cancer, lipid abnormalities, cardiovascular disease and psychological abnormalities, male pattern baldness (alopecia), benign prostatic hyperplasia (BPH) and acne, hirsutism, amenorrhea, hypogonadism, anemia, diabetes, defects in spermatogenesis, cachexia, osteoporosis, osteopenia, and muscle wasting.

Methods of treating such diseases by administering a therapeutically effective amount of such compounds to a patient are also provided by the invention.

The compounds of the present invention can be used alone, in combination with other compounds of the present invention, or in combination with one or more other agent(s) active in the therapeutic areas described herein.

According to another aspect of the invention there is provided a compound as defined in Formula I, provided that the compound is not the compound according to the formula;

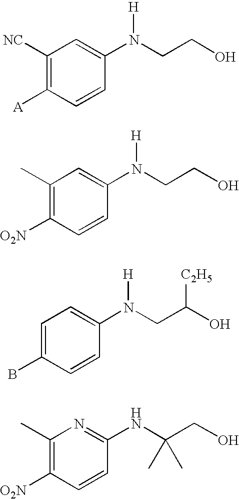

wherein A is —CN or —NO₂, and B is —CN, —NO₂ or —SO₂CH₃.

The compounds above are known in the prior art as an intermediate compound in the manufacture of compounds used in different technical fields, namely the dye industry, or herbicide, or the compound synthesis has merely been reported with no application disclosed (Compound Reference: Specs and Bio Specs B.V.; Catalog No. AK-079/11126007; EP797980; U.S. Pat. No. 4,723,986; DE 2331900; and Zorihe et al; Doklad, Akadenii Náuk, SSSR (1989), 208(5), (1150-1154).

### DETAILED DESCRIPTION OF THE INVENTION

The following definitions apply to the terms as used throughout this specification, unless otherwise limited in specific instances.

The term “androgen receptor ligand” as used herein is intended to cover any moiety, which binds to an androgen receptor. The ligand may act as an antagonist, or as a partial antagonist.

A compound being a “partial antagonist” is a compound with both agonistic and antagonistic activity.

The term “alkyl” as employed herein alone or as part of another group refers to an acyclic straight or branched chain radical, containing 1 to about 10 carbons, preferably 1 to 6 carbons in the normal chain, i.e. methyl, ethyl, propyl, isopropyl, sec-butyl, tert-butyl, pentyl, hexyl, octyl. When substituted alkyl is present, this refers to an unbranched or branched alkyl group, which groups may be the same or different at any available point, as defined with respect to each variable.

The term “substituted alkyl” includes an alkyl group optionally substituted with one or more functional groups which are commonly attached to such chains, such as, alkyl, alkenyl, alkynyl, aryl, cycloalkyl, heteroaryl, hydroxy, cyano, nitro, amino, halo, carboxyl or alkyl ester thereof and/or carboxamide.

The term “alkenyl” as employed herein alone or as part of another group refers to a straight or branched chain radical, containing 2 to about 10 carbons, preferably 2 to 6 carbons i.e. ethenyl, propenyl, butenyl, allyl.

The term “allyl” refers to H₂C═CH—CH₂.

The term “alkynyl” as employed herein alone or as part of another group refers to a straight or branched chain radical, containing 2 to about 10 carbons, preferably 2 to 6 carbons i.e. ethynyl, propynyl, butynyl, allyl.

The term “aryl” as employed herein alone or as part of another group refers to substituted and unsubstituted aromatic ring system. The terms aryl includes monocyclic aromatic rings, polycyclic aromatic ring system and polyaromatic ring systems. The polycyclic aromatic and polyaromatic ring systems may contain from two to four, more preferably two to three rings. Preferred aryl groups include 5- or 6-membered ring systems.

The term “heteroaryl” refers to optionally substituted aromatic ring system having one or more heteroatoms such as, for example, oxygen, nitrogen and sulfur. The terms heteroaryl includes five- or six-membered heterocyclic rings, polycyclic heteroaromatic ring system and polyheteroaromatic ring systems. The poly heterocyclic aromatic and poly heteroaromatic ring systems may contain from two to four, more preferably two to three rings. The term hetero aryl includes ring system such as pyridine, quinoline, furan, thiophene, pyrrole, imidazole and pyrazole.

The term “alkoxy” as employed herein alone or as part of another group refers to an alkyl ether wherein the term alkyl is as defined above. Examples of alkoxy radicals include methoxy, ethoxy, n-propoxy, isopropoxy, n-butoxy and the like.

The term “aryloxy” as employed herein alone or as part of another group refers to an aryl alkyl ether, wherein the term aryl is as defined above. Examples of aryloxy radicals include phenoxy, benzyloxy and the like.

The term “alkylthio” as employed herein alone or as part of another group refers to an alkyl thio wherein the term alkyl is as defined above and one of the methylene carbons has been replaced with sulfur. Examples of alkylthio radicals include methane thiol, ethane thiol, propane thiol, —(CH2)ₘS(CH2)ₙ, wherein m+n=9 and the like.

The term “alkylsulphone” as employed herein alone or as part of another group refers to an alkylsulphone wherein the term alkyl is as defined above and one of the methylene carbons has been replaced with sulfur. Examples of alkylsulphone radicals include methanesulphone, ethanesulphone, propanesulphone, —(CH2)ₘSO2(CH2)ₙ, where m+n=9 and the like.

The term “alkylsulphoxide” as employed herein alone or as part of another group refers to an alkylsulphoxide wherein the term alkyl is as defined above and one of the methylene carbons has been replaced with sulfur. Examples of alkylsulphoxide radicals include methanesulphoxide, ethanesulphoxide, propanesulphoxide —(CH2)ₘSO(CH2)ₙ, where m+n=9 and the like.

The term “alkylarylthio” as employed herein alone or as part of another group refers to an arylalkylthio wherein the term alkylthio and aryl are as defined above and one of the terminal methyl groups is substituted with aryl. Examples of —(CH2)ₘS(CH2)ₙ, CH2—Ar where m+n=8 and the like.

The term “alkylarylsulphone” as employed herein alone or as part of another group refers to an arylalkylsulphone wherein the term alkylsulphone and aryl are as defined above and one of the terminal methyl groups is substituted with aryl. Examples of —(CH2)ₘSO2(CH2)ₙ, CH2—Ar where m+n=8 and the like.

The term “alkylarylsulphoxide” as employed herein alone or as part of another group refers to an arylalkysulphoxide wherein the term alkylsulphoxide and aryl are as defined above and one of the terminal methyl groups is substituted with aryl. Examples of —(CH2)ₘSO(CH2)ₙ, CH2—Ar where m+n=8 and the like.

The term “cycloalkyl” as employed herein alone or as part of another group refers to saturated cyclic hydrocarbon groups or partially unsaturated cyclic hydrocarbon groups, independently containing one carbon-to-carbon double bond. The cyclic hydrocarbon contains 3 to 4 carbons. It should also be understood that the present invention also involve cycloalkyl rings where 1 to 2 carbons in the ring are replaced by either —O—, —S— or —N—, thus forming a saturated or partially saturated heterocycle. Examples of such rings are aziridine, thiiranes and the like. Preferred heterocyclic rings are 3-membered, which may be optionally substituted by 1, 2 or 3 groups of R^{a} which groups may be the same or different through available carbons as in the case of “alkyl”. Preferred cycloalkyl groups include 3 carbons, such as cyclopropyl, which may be optionally substituted by 1, 2 or 3 groups of R^{a} which groups may be the same or different through available carbons as in the case of “alkyl”.

The term “halogen” refers to fluorine, chlorine, bromine and iodine. Also included are carbon substituted halogens such as —CF₃, —CHF₂, and —CH₂F.

The compounds of the present invention can be present as salts, which are also within the scope of this invention. Pharmaceutically acceptable (i.e., non-toxic, physiologically acceptable) salts are preferred. If the compounds of the invention have, for example, at least one basic center, they can form acid addition salts. These are formed, for example, with strong inorganic acids, such as mineral acids, for example sulfuric acid, phosphoric acid or a hydrohalic acid, with strong organic carboxylic acids, such as alkanecarboxylic acids of 1 to 4 carbon atoms which are unsubstituted or substituted, for example, by halogen, for example acetic acid, such as saturated or unsaturated dicarboxylic acids, for example oxalic, malonic, succinic, maleic, fumaric, phthalic or terephthalic acid, such as hydroxycarboxylic acids, for example, ascorbic, glycolic, lactic, malic, tartaric or citric acid, such as amino acids, (for example aspartic or glutamic acid or lysine or arginine), or benzoic acid, or with organic sulfonic acids, such as (C₁-C₄) alkyl or arylsulfonic acids which are unsubstituted or substituted, for example by halogen, for example methyl- or p-toluene-sulfonic acid. Corresponding acid addition salts can also be formed having, if desired, an additionally present basic center. The compounds of the invention having at least one acid group (e.g. COOH) can also form salts with bases. Suitable salts with bases are, for example, metal salts, such as alkali metal or alkaline earth metal salts, for example sodium, potassium or magnesium salts, or salts with ammonia or an organic amine, such as morpholine, thiomorpholine, piperidine, pyrrolidine, a mono, di or tri-lower alkylamine, for example ethyl, tertbutyl, diethyl, diisopropyl, triethyl, tributyl or dimethyl-propylamine, or a mono, di or trihydroxy lower alkylamine, for example mono, di or triethanolamine. Corresponding internal salts may furthermore be formed. Salts that are unsuitable for pharmaceutical uses but which can be employed, for example, for the isolation or purification of free compounds of the invention or their pharmaceutically acceptable salts, are also included. Preferred salts of the compounds of the present invention which contain a basic group include monohydrochloride, hydrogensulfate, methanesulfonate, phosphate or nitrate. Preferred salts of the compounds of formula I which contain an acid group include sodium, potassium and magnesium salts and pharmaceutically acceptable organic amines.

The compounds according to the invention may also have prodrug forms. Any compound that will be converted in vivo to provide the bioactive agent (i.e., the compound of formula I) is a prodrug within the scope and spirit of the invention. Such prodrugs are well known in the art and a comprehensive description of these may be found in: (i) The Practice of Medicinal Chemistry, Camille G. Wermuth et al., Ch 31, (Academic Press, 1996); (ii) Design of Prodrugs, edited by H. Bundgaard, (Elsevier, 1985); and (iii) A Textbook of Drug Design and Development, P. Krogsgaard-Larson and H. Bundgaard, eds. Ch 5, pgs 113-191 (Harwood Academic Publishers, 1991).

Embodiments of prodrugs suitable for use in the present invention include lower alkyl esters, such as ethyl ester, or acyloxyalkyl esters such as pivaloyloxymethyl (POM).

The compounds according to the present invention are preferably administered in a therapeutically effective amount. The term “therapeutically effective amount” as used herein refers to an amount of a therapeutic agent to treat or prevent a condition treatable by administration of a composition of the invention. That amount is the amount sufficient to exhibit a detectable therapeutic or preventative or ameliorative effect. The effect may include, for example, treatment or prevention of the conditions listed herein. The precise effective amount for a subject will depend upon the subject's size and health, the nature and extent of the condition being treated, recommendations of the treating physician, and the therapeutics or combination of therapeutics selected for administration.

Scheme 1-6 outlines the synthetic routes used for preparing the compound according to Formula I.

### Scheme 1

Synthetic routes to these compounds can be visualized by the skilled person and the present synthetic route is not limiting for the invention. 4-Fluro-1-nitro-2-trifluromethyl-benzene (1a) and 4-fluoro-2-methyl-1-nitro-benzene (1b) were employed as starting material in scheme-1 and is commercially obtainable.

Scheme 1 depicts a synthesis of compounds of formula I in which R₆ is CF₃ and Me and is connected to phenyl ring. Condensation of compound (1a) with different β-amino alcohols and di-isopropyl ethylamine in DMSO gave compound 3 (examples 1-4) in quantitative yield. The reactions were performed in a microwave oven at elevated temperature for a short time. Compound (1b) was used for producing the compound 3 (examples 5-7) and similar conditions were adopted as in examples 1-4. An alternative method was used for the preparation of example-5. The reaction according to the alternative method was performed by heating the compound (1b) and β-amino alcohol in pentanol in a sealed tube.

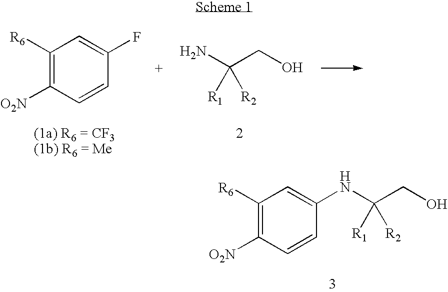

### Scheme 2

Compounds 9 (examples 8-15) were prepared from starting material 6-chloro-3-nitro-2-picoline (compound 4). Starting material was synthesized in three steps starting with compound 6-amino-2-picoline using the literature procedure. Nitration of 6-amino-2-picoline was accomplished by concentrated sulphuric acid (H₂SO₄) and concentrated nitric acid (HNO₃) and provided 6-amino-3-nitro-2-picoline (Baumgarten, H. E. and Chien Fan Su, H. JACS 74 (1952) 3828; Parker, E. D. and Shive, W. JACS 69 (1947) 63). Treatment of 6-amino-3-nitro-2-picoline with sodium nitrite provided 6-hydroxy-3-nitro-2-picoline, which, when reacted with PCl₅ and POCl₃, provided 6-chloro-3-nitro-2-picoline (Baumgarten, H. E. and Chien Fan Su, H. JACS 74 (1952) 3828).

Scheme 2 shows the synthesis of compounds of formula I in which Z is N and R₇ is H. Condensation of 6-Chloro-3-nitro-2-picoline and 2-amino-2-methyl-propan-1-ol in 1-pentanol and the mixture refluxed under inert atmosphere gave compound 9 (example-8) as yellow crystals. 6-Chloro-3-nitro-2-picoline can also be purchased as commercial starting material. The reaction time was reduced by using a microwave oven. Condensation of compound 7 with different β-amino alcohols (8) in the microwave provided compound 9 (examples 9-13) in quantitative yield. Synthetic routes to these compounds can be visualized by the skilled person. Reaction of compound (10) with L-alanine provided compound 11 (example-1 4). Reduction of the acid compound (11) by a reducing agent such as lithium aluminum hydride (LAH) produced compound 9 (example 15).

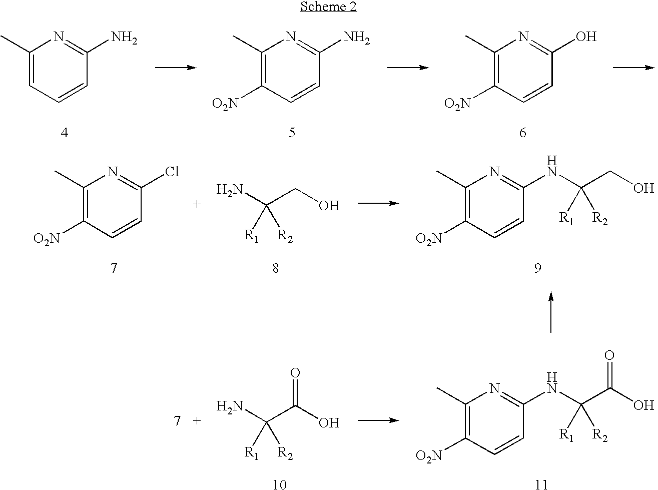

### Scheme 3

Synthesis of compounds according to formula I, in which R₆ and R₇ are Me and connected to the phenyl ring is shown in Scheme-3. 4-Fluoro-2,3-di-methyl-1-nitro-benzene (13) was employed as starting material in scheme-3, which was produced by the nitration of compound (12) with fuming nitric acid in acetic anhydride in one step. Condensation of 2,3-dimethyl-fluoro-benzene with β-amino alcohols at higher temperature gave compound 14 (example 16).

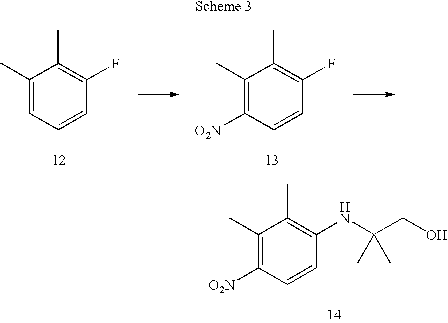

### Scheme 4

Scheme 4 depicts a synthesis of compounds of formula I in which R₆ and R₈ are Me and connected to the phenyl ring. Condensation of compound (15) with (S)-2-amino-butan-1-ol and di-isopropyl ethylamine in DMSO gave compound 16 (examples 17). The reaction was performed in a microwave oven.

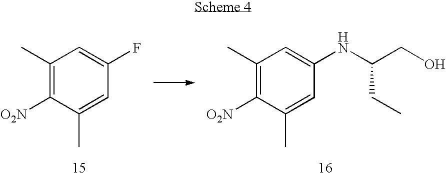

### Scheme 5

Reduction of nitro compound to amine was accomplished by the treatment of sodium thiosulphate with ethanol. After work-up the amines were used for the next step without any further purification. Reaction of amine with potassium cyanide and copper cyanide in water gave compound 19 (examples 26-28). (Clive, D. L. et al. JOC 52 (1987) 1339-42 and Vogel expt. 6.76). Some other examples of compound 19 were made by an alternative method utilizing a microwave oven. Similar reaction conditions as those used in scheme-1 and scheme-2 provided compound 19 (examples 18-22).

Conversion of the nitrile form of compound 19 to benzoic acid compound 20 (example 87) was performed in a refluxed aqueous sodium hydroxide solution in methanol.

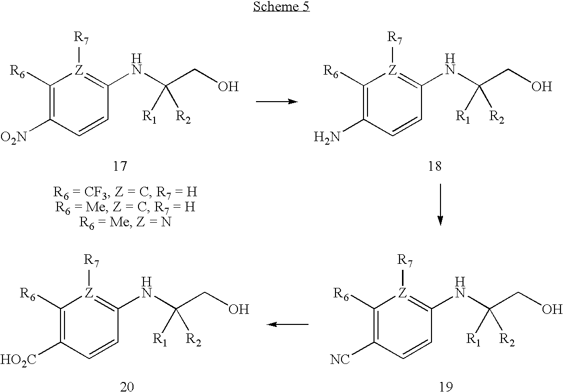

### Scheme 6

Scheme 6 depicts a synthesis of compounds of formula I in which R₃ and R₄ are Me and is connected to the alkyl chain. Condensation of 6-chloro-3-nitro-2-picoline with glycine methyl ester hydrochloride and triethyl amine in DMSO gave compound 22 (example 88). Compound 22 was treated with methyl magnesium bromide and after HPLC purification gave compound 23 (example 89).

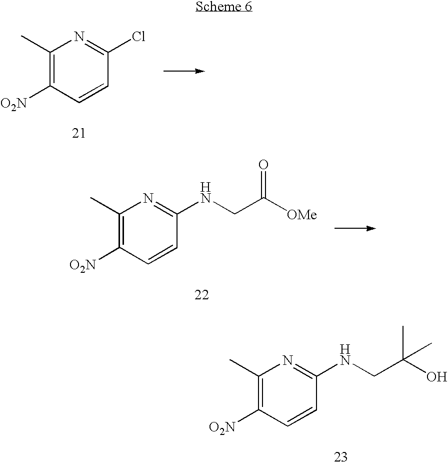

### EXAMPLES

The following Examples represent preferred embodiments of the present invention. However, they should not be construed as limiting the invention in any way. The ¹H NMR spectra were consistent with the assigned structures. Mass spectra were recorded on a Perkin-Elmer, API 150Ex spectrometer, with turbo “ion spray” on negative ion mode (ES−1) or positive (ES+1), using a Zorbax SB-C8 column (LC-MS). The microwave reactions were performed in a Personal Chemistry Emrys Optimizer.

### Example 1

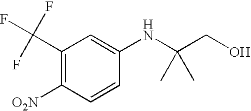

### 2-Methyl-2-(4-nitro-3-trifluoromethyl-phenylamino)-propan-1-ol

4-Fluoro-1-nitro-2-trifluoromethyl-benzene (1.226 g, 5.86 mmol) was dissolved in 7 mL DMSO and 2-amino-2-methyl-propan-1-ol (784 mg, 8.795 mmol) was added, followed by diisopropyl ethylamine (DIPEA) (985 mg, 7.622 mmol). The reaction was heated to 180° C. for 900 seconds in a microwave oven (Parameters: high absorbance, fixed holding time, pre-stirring 25 seconds). The mixture was diluted with 20 mL of EtOAc and then washed three times with an aqueous solution of ammonium chloride (NH₄Cl). The organic phase was collected, dried with MgSO₄ (anhydrous) and filtered. The dry organic phase was evaporated in vacuo. The crude product was a bright yellow powder. The crude product was purified on a silica column with 5:1 n-heptane:EtOAc as mobile phase. This gave 1.1 g (68%) of 2-methyl-2-(4-nitro-3-trifluoromethyl-phenylamino)-propan-1-ol as a yellow solid. M/Z=278

### Example 2

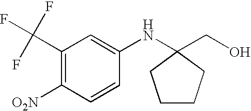

### [1-(4-Nitro-3-trifluoromethyl-phenylamino)-cyclopentyl]-methanol

4-Fluoro-1-nitro-2-trifluoromethyl-benzene (122 mg, 0.583 mmol) was coupled with (1-amino-cyclopentyl)-methanol (101 mg, 0.875 mmol), DIPEA (90.5 mg, 0.700 mmol) in DMSO 0.8 mL, using the same procedure as described in Example-1. This gave 120.5 mg (68%) of [1-(4-nitro-3-trifluoromethyl-phenylamino)-cyclopentyl]-methanol as a yellow powder. M/Z=304.

### Example 3

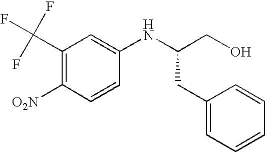

### (S)-2-(4-Nitro-3-trifluoromethyl-phenylamino)-3-phenyl-propan-1-ol

4-Fluoro-1-nitro-2-trifluoromethyl-benzene (119 mg, 0.569 mmol) was coupled with (S)-2-amino-3-phenyl-propan-1-ol (129 mg, 0.854 mmol), DIPEA (88 mg, 0.683 mmol) in DMSO 0.8 mL using the same procedure as described in Example-1. This gave 112 mg (58%) of (S)-2-(4-nitro-3-trifluoromethyl-phenylamino)-3-phenyl-propan-1-ol as yellow crystals. M/Z=340.

### Example 4

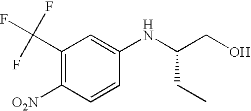

### (S)-2-(4-Nitro-3-trifluoromethyl-phenylamino)-butan-1-ol

4-Fluoro-1-nitro-2-trifluoromethyl-benzene (122 mg, 0.583 mmol) was coupled with (S)-2-amino-butan-1-ol (78 mg, 0.875 mmol), DIPEA (91 mg, 0.700 mmol) in DMSO 0.8 mL using the same procedure as described in Example-1. This gave 107 mg (67%) of (S)-2-(4-nitro-3-trifluoromethyl-phenylamino)-butan-1-ol as yellow oily crystals. M/Z=278.

### Example 5

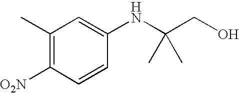

### 2-Methyl-2-(3-hydroxy-4-nitro-phenylamino)-propan-1-ol

Method-A: 4-Fluoro-2-methyl-1-nitro-benzene (113 mg, 0.728 mmol) was coupled with 2-amino-2-methyl-propan-1-ol (84 mg, 0.947 mmol), DIPEA (122 mg, 0.947 mmol) in DMSO 1.2 mL using the same procedure as described in Example-1. The crude product was purified on a silica column with 1:1 n-heptane:EtOAc as mobile phase. This gave 72 mg (44%) of 2-methyl-2-(3-methyl-4-nitro-phenylamino)-propan-1-ol as yellow powder. M/Z=224.

Method-B: 4-Fluoro-2-methyl-1-nitro-benzene (2.33 g, 15 mmol) and 2-amino-2-methylpropanol (2.67 g, 30 mmol) were heated with stirring at 160° C. in a sealed tube overnight. The reaction mixture was diluted with EtOAc and purified by flash chromatography (dry application; 14% EtOAc in hexane→EtOAc) to afford 2.85 g (85%) of the 2-methyl-2-(3-hydroxy-4-nitro-phenylamino)-propan-1-ol.

### Example 6

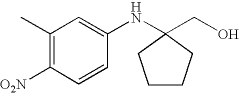

### [1-(3-Methyl-4-nitro-phenylamino)-cyclopentyl]-methanol

4-Fluoro-2-methyl-1-nitro-benzene (107 mg, 0.689 mmol) was coupled with (1-amino-cyclopentyl)-methanol (103 mg, 0.897 mmol), DIPEA (116 mg, 0.897 mmol) in DMSO 1.2 mL using the same procedure as described in Example-1. The crude product was purified on a silica column with 1:1 n-heptane:EtOAc as mobile phase. This gave 76 mg (44%) of [1-(3-methyl-4-nitro-phenylamino)-cyclopentyl]-methanol as a yellow powder. M/Z=250.

### Example 7

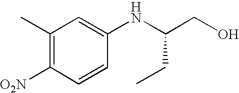

### (S)-2-(3-Methyl-4-nitro-phenylamino)-butan-1-ol

4-Fluoro-2-methyl-1-nitro-benzene (102 mg, 0.658 mmol) was coupled with (S)-2-amino-butan-1-ol (76 mg, 0.855 mmol), DIPEA (111 mg, 0.855 mmol) in DMSO 1.2 mL using the same procedure as described in Example-1. The crude product was purified on a silica column with 1:1 n-heptane:EtOAc as mobile phase. This gave 85 mg (58%) of (S)-2-(3-methyl-4-nitro-phenylamino)-butan-1-ol as yellow oil. M/Z=224.

### Example 8

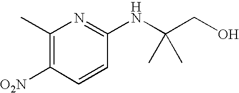

### 2-Methyl-2-(6-methyl-5-nitro-pyridin-2-ylamino)-propan-1-ol

(a) Conc. H₂SO₄ (140ml) was cooled in an ice-salt bath and molten 6-amino-2-picoline (30 g, 0.277 mol) was added in portions with good stirring. To this brown, viscous solution which was maintained at 0° C. was added a cooled (0° C.) mixture of conc. H₂SO₄ (21 ml) and conc. HNO₃ (21 ml) drop wise over a period of approx. 1.5 hrs. The red-orange reaction mixture was stirred for an additional hour at 0° C. and then allowed to warm slowly to room temperature over night. The brown solution was heated at 60° C. (oil bath) for 1 hr followed by 1 hr at 100° C. (carefully controlled temperature). The reaction mixture was cooled to 0° C. (ice bath), poured over cracked ice and neutralised by addition of a concentrated aqueous NaOH solution. The yellow precipitate was filtered and washed well with ice-water. (The filtrate was put in the refrigerator; additional product was precipitated together with the salts.) The yellow product was suspended in water and divided into two portions, each of them subjected to steam distillation in turn. The yellow reaction mixture became more “transparent” after some hrs, but the collected steam, containing 4-amino-3-nitro-2-picoline, was still yellow after 6 hrs. The steam distillation was stopped after 8 hrs, the residual part of the reaction mixture was filtered and evaporated to dryness. ¹HNMR (D₂O) showed a mixture of 2-3 compounds. The mixture was washed with; CHCl₃, EtOH (x 2) and CHCl₃ leaving 20.4 g (48%) of pure 6-amino-3-nitro-2-picoline.

(b) 6-Amino-3-nitro-2-picoline (20 g, 0.131 mol) was suspended in a mixture of conc. H₂SO₄ (23.7 ml) and water (335 ml). More conc. H₂SO₄ (20 ml) was added under ice-cooling, but the amine did not dissolve completely. The suspension was added in ice (100 g) before a solution of NaNO₂ (13.53 g, 0.196 mol) in water (40 ml) was added drop wise. Gas evolution was observed. The brown suspension was stirred at 10° C. for 1 hr, filtered and washed with water. The brown product was dried (freeze dryer) to achieve 15.78 g (78%) of 6-hydroxy-3-nitro-2-picoline.

(c) To 6-Hydroxy-3-nitro-2-picoline (15.73 g, 0.102 mol) was added PCl₅ (5.73 g, 0.027 mol) and POCl₃ (2.9 ml, 0.032 mol). This mixture was heated at 110-115° C. for 3 hrs. However, the amount of POCl₃ added was only enough to moisten the starting material. More POCl₃ (3 ml) was added, the reaction mixture heated at 110-115° C. but only sublimation of PCl₅ (100° C.) was observed. DMF (5 ml) was added and the solution was heated at 115° C. for 5 hrs, cooled and poured into a slush of ice and water. A beige product precipitated and the water suspension was stirred for 48 hrs. The brown precipitate was filtered off and washed with water. Purification by dry-flash dichloromethane yielded 10.93 g (62%) of 6-chloro-3-nitro-2-picoline.

(d) 6-Chloro-3-nitro-2-picoline (6.055 g, 35.1 mmol) and 2-amino-2-methyl-propan-1-ol (6.2 g, 73.7 mmol) were suspended in 1-pentanol (30 ml) and the mixture refluxed under inert atmosphere overnight. The thin layer chromatography (dichloromethane 4/EtOAc 1) revealed some remaining starting material, so the reaction was refluxed for another 3.5 hrs. The reaction mixture was cooled and water was added under stirring. A sticky, yellow precipitate was filtered off, washed well with water and dried. The crude product (6.04 g) was re-crystallised from either pentane-acetone or dichloromethane. Collecting the crops furnished 5.71 (72%) of 2-methyl-2-(6-methyl-5-nitro-pyridin-2-ylamino)-propan-1-ol as yellow crystals. M/Z 225.

### Example 9

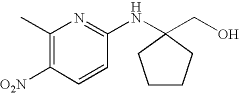

### [1-(6-Methyl-5-nitro-pyridin-2-ylamino)-cyclopentyl]-methanol

6-Chloro-3-nitro-2-picoline (22 mg, 0.13 mmol) was coupled with (1-amino-cyclopentyl)-methanol (31 mg, 0.27 mmol), triethylamine (0.025 mL, 0.18 mmol) in 2-pentanol (1 mL). The reaction was heated to 180° C. for 2 h in a microwave oven (Parameters: high absorbance, fixed holding time, pre-stirring 25 seconds). The mixture was diluted with 20 mL of EtOAc and then washed with NaHCO₃. The organic phase was collected, dried with anhydrous MgSO₄ and filtered. The dry organic phase was evaporated and purified on a silica column with 5:1 n-Heptane:EtOAc as mobile phase. This gave 9 mg (28%) of [1-(6-methyl-5-nitro-pyridin-2-ylamino)-cyclopentyl]-methanol as a yellow solid. M/Z=251

### Example 10

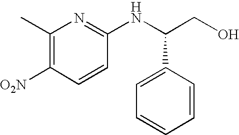

### (S)-2-(6-Methyl-5-nitro-pyridin-2-ylamino) 2-phenyl-ethanol

6-Chloro-3-nitro-2-picoline (22 mg, 0.13 mmol) was coupled with (2-amino-2-phenyl)-propanol (34 mg, 0.25 mmol) in triethylamine (0.030 mL, 0.25 mmol) in DMSO (1 mL). The reaction was heated to 140° C. for 1200 seconds in a microwave oven (Parameters: high absorbance, fixed holding time, pre-stirring 25 seconds). The mixture was diluted with 20 mL of EtOAc and then washed with NH₄Cl (aq) three times. The organic phase was collected, dried with anhydrous MgSO₄ and filtered. The dry organic phase was evaporated and purification on silica column with 5:1 n-Heptane:EtOAc gave 22 mg (63%) of (R)-2-(6-methyl-5-nitro-pyridin-2-ylamino) 2-phenyl-ethanol as a yellow solid. M/Z=273.

### Example 11

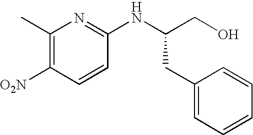

### (S)-2-(6-Methyl-5-nitro-pyridin-2-ylamino)-3-phenyl-propan-1-ol

6-Chloro-3-nitro-2-picoline (30 mg, 0.17 mmol) was coupled with (S)-2-amino-3-phenyl-propan-1-ol (32 mg, 0.21 mmol), sodium acetate (28 mg, 0.34 mmol) in EtOH (2 mL). The reaction was heated in a microwave oven for 20 min at 130° C. and then additionally 20 minutes at 150° C. The reaction was quenched with a saturated aqueous solution of NaHCO₃ and extracted with EtOAc and evaporated. Purification on a silica column with a gradient solution of heptane:EtOAc gave 24 mg (48%) of (S)-2-(6-methyl-5-nitro-pyridin-2-ylamino)-3-phenyl-propan-1-ol as a yellow solid. M/Z=287.

### Example 12

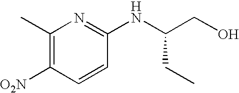

### (S)-2-(6-Methyl-5-nitro-pyridin-2-ylamino)-butan-1-ol

6-Chloro-3-nitro-2-picoline (30 mg, 0.17 mmol) was coupled with (S)-2-amino-butan-1-ol (32 mg, 0.21 mmol), and sodium acetate (28 mg, 0.34 mmol) in EtOH (2 mL) using the same procedure as described in Example-13. This gave 21 mg (53%) of (S)-2-(6-methyl-5-nitro-pyridin-2-ylamino)-butan-1-ol as a yellow solid. M/Z=225.

### Example 13

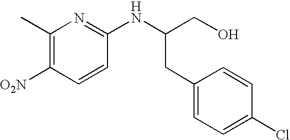

### (DL)-3-(4-Chloro-phenyl)-2-(6-methyl-5-nitro-pyridin-2-ylamino)-propan-1-ol

6-Chloro-3-nitro-2-picoline (50 mg, 0.29 mmol) was coupled with (DL)-2-amino-3-(4-chloro-phenyl)-propan-1-ol (103 mg, 0.55 mmol), in triethylamine (0.077 mL, 0.55 mmol) in DMSO (1 mL) using the same procedure as described in Example-1 but at 140° C. This gave 23 mg (45%) of (DL)-2-(6-methyl-5-nitro-pyridin-2-ylamino)-3-(4-chloro-phenyl)-propan-1-ol as a yellow solid. M/Z=321.

### Example 14

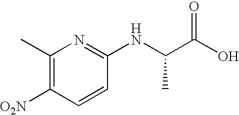

### (S)-2-(6-Methyl-5-nitro-2-pyridin-2-ylamino)-propionic acid

6-Chloro-3-nitro-2-picoline (62 mg, 0.36 mmol) was coupled with L-alanine (80 mg, 0.90 mmol) and sodium acetate (78 mg, 0.95 mmol) in DMSO 1 mL. The reaction was heated to 140° C. for 600 seconds in a microwave oven (Parameters: high absorbance, fixed holding time, pre-stirring 25 seconds). The crude mixture was treated with a saturated aqueous solution of NH₄Cl. The reaction mixture was acidified to pH 4 (HCl, 1M). The crude reaction mixture was extracted with EtOAc, and the combined organic layers were washed with water and brine. Purification on silica using a mobile phase CH₂Cl₂—MeOH—HOAc gave 60 mg (74%) of (S)-2-(6-methyl-5-nitro-2-pyridin-2-ylamino)-propionic acid as a yellow solid. M/Z=225.

### Example 15

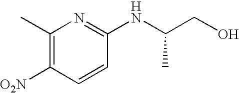

### (S)-2-(6-Methyl-5-nitro-pyridin-2-ylamino)-propan-1-ol

(S)-2-(6-Methyl-5-nitro-pyridin-2-ylamino)-propionic acid (60 mg, 0.27 mmol) was added to a nitrogen-purged flask with LiAlH₄ (27 mg, 0.71 mmol). The reaction mixture was refluxed for 2 h and then allowed to reach room temperature and then quenched by sequentially adding H₂O (1 mL), NaOH (1M, 1 mL) and H₂O (1 mL). The slurry was centrifuged and the precipated aluminum salts were washed with dichloromethane. The combined filtrates were evaporated and purification of the residue on a silica column with heptane-EtOAc (3:2) gave 13 mg (22%) of (S)-2-(6-methyl-5-nitro-pyridin-2-ylamino)-propan-1-ol as a yellow solid. M/Z=211.

### Example 16

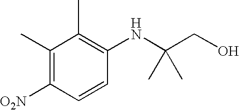

### 2-(2,3-Dimethyl-4-nitro-phenylamino)-2-methyl-propan-1-ol

Fuming nitric acid (1.4 g, 20.3 mmol) was cooled to 0° C. and acetic anhydride (2.89 g, 28.4 mmol) was added. This solution was added to a cold (0° C.) solution of 3-fluoro-1,2-dimethylbenzene (1.0 g, 8.1 mmol) in acetic anhydride (4 ml) over 10 min. The reaction mixture was stirred for 25 min, poured slowly over ice and the water solution extracted with EtOAc (x 3). The collected organic phase was washed with diluted saturated aqueous solution of NaHCO₃ followed by brine before evaporation to dryness. The residue was flash purified on a silica gel column using hexane as a mobile phase to give 2,3-dimethyl-4-fluoro-1-nitro-benzene 0.74 g (54%) as a yellow oil which crystallised upon standing.

The fluoride (0.576 g, 3.4 mmol) was mixed with 2-amino-2-methylpropanol (0.61 g, 6.8 mmol) in a tube, and the tube was sealed before immersing it into an oil bath and heating at 160° C. for 5 days. TLC (Hexane) showed remaining starting material. The reaction mixture was cooled and diluted with EtOAc before purification by flash silica gel chromatography (dry application; 6:4 hexane and EtOAc) to give 0.34 g (59% recovery) of the starting material 2,3-dimethyl-4-fluoro-1-nitro-benzene and 0.20 g (61% based on recovered starting material) of the 2-(2,3-dimethyl-4-nitro-phenylamino)-2-methyl-propan-1-ol. M/Z=238.

### Example 17

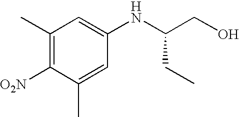

### (S)-2-(3,5-Dimethyl-4-nitro-phenylamino)-butan-1-ol

(S)-2-Amino-butan-1-ol (41 mg, 0.461 mmol) was dissolved in DMSO (800 μL) and DIPEA (80 μL, 0.461 mmol) added. 4-Fluoro-2-trifluoromethyl-benzonitrile (60mg, 0.354 mmol) was added and the reaction mixture was heated to 160° C. for 900 seconds in a microwave oven (Parameters: High absorbance, Fixed Holding time, pre-stirring 25 sec). The reaction mixture was then diluted with EtOAc and washed with an aqueous solution of NH₄Cl. The organic phase was then dried and evaporated in vacuo. The crude product was purified on silica column with 3:1 n-heptane:EtOAc as the mobile phase. This provided 22 mg (26%) of (S)-2-(3,5-dimethyl-4-nitro-phenylamino)-butan-1-ol. M/Z=238

### Example 18

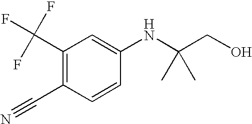

### 4-(2-Hydroxy-1,1-dimethyl-ethylamino)-2-trifluoromethyl-benzonitrile

2-Amino-2-methyl-propan-1-ol (25 mg, 0.275 mmol) was dissolved in 0.7 mL DMSO and DIPEA (36 mg, 0.275 mmol) was added. 4-fluoro-2-trifluoromethyl-benzonitrile (40 mg, 0.212 mmol) was then added and the reaction was heated to 140° C. for 1100 seconds in a microwave oven (Parameters: high absorbance, fixed holding time, pre-stirring 25 seconds). The reaction was then diluted with 10 mL EtOAc, washed with an aqueous solution of NH₄Cl, dried with anhydrous MgSO₄, filtered and then the organic phase was evaporated in vacuo. The crude product was purified on silica column with 3:1 n-heptane:EtOAc as the mobile phase. Upon dissolving the crude product in the mobile phase, an insoluble precipitate was collected. On analysis this showed to be mainly pure product. All insoluble precipitate was dissolved in acetone, celite™ was added, whereafter the acetone was evaporated. The celite was then applied to a silica column with 2:1 n-heptane:EtOAc as the mobile phase to give 34 mg (62%) of 4-(2-hydroxy-1,1-dimethyl-ethylamino)-2-trifluoromethyl-benzonitrile as beige crystals. M/Z=258.

### Example 19

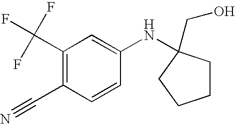

### 4-(1-Hydroxymethyl-cyclopentylamino)-2-trifluoromethyl-benzonitrile

4-Fluoro-2-trifluoromethyl-benzonitrile (40 mg, 0.212 mmol) was coupled with (1-amino-cyclopentyl)-methanol (32 mg, 0.275 mmol), and DIPEA (36 mg, 0.275 mmol) in DMSO 0.7 mL using the same procedure as described in Example-8. This gave 23 mg (38%) of 4-(1-hydroxymethyl-cyclopentylamino)-2-trifluoromethyl-benzonitrile as white powder. M/Z=284.

### Example 20

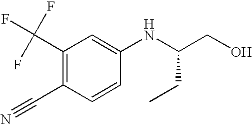

### (S)-4-(1-Hydroxymethyl-cyclopentylamino)-2-trifluoromethyl-benzonitrile

4-Fluoro-2-trifluoromethyl-benzonitrile (40 mg, 0.212 mmol) was coupled with (S)-2-amino-butan-1-ol (25 mg, 0.275 mmol), DIPEA (36 mg, 0.275 mmol), in 0.7 mL DMSO using the same procedure as described in Example-8. This gave 17 mg (31%) of (S)-4-(1-hydroxymethyl-cyclopentylamino)-2-trifluoromethyl-benzonitrile as white crystals. M/Z=258.

### Example 21

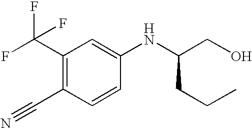

### (R)-4-(1-Hydroxymethyl-butylamino)-2-trifluoromethyl-benzonitrile

4-Fluoro-2-trifluoromethyl-benzonitrile (40 mg, 0.21 mmol), (R)-2-Amino-pentan-1-ol (32 mg, 0.27 mmol) and DIPEA (47 μL, 0.27 mmol) was dissolved in DMSO (1 mL) and heated to 180° C. for 900 seconds in a microwave oven (Parameters: Fixed Holding time, High absorbance, pre-stirring 25 sec.). The crude product was diluted with CH₂Cl₂ and washed with an aqueous solution of NH₄Cl. The organic phase was separated, dried and evaporated in vacuo. The crude product was purified on a silica column with 3:1 n-heptane:EtOAc as the mobile phase. This gave 39 mg (68%) of (R)-4-(1-hydroxymethyl-butylamino)-2-trifluoromethyl-benzonitrile. M/Z=272.

### Example 22

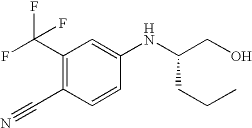

### (S)-4-(1-Hydroxymethyl-butylamino)-2-trifluoromethyl-benzonitrile

4-Fluoro-2-trifluoromethyl-benzonitrile (40 mg, 0.21 mmol) was coupled with (S)-2-Amino-pentan-1-ol (32 mg, 0.27 mmol), DIPEA (47 μL, 0.27 mmol) in DMSO 1.0 mL, using the same procedure as described in Example-21. This gave 24 mg (42%) of (S)-4-(1-hydroxymethyl-butylamino)-2-trifluoromethyl-benzonitrile. M/Z=272

### Example 23

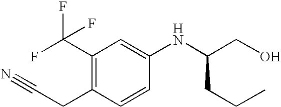

### [4-(R)-1-Hydroxymethyl-butylamino)-2-trifluoromethyl-phenyl]-acetonitrile

(4-Fluoro-2-trifluoromethyl-phenyl)-acetonitrile (100 mg, 0.492 mmol) was dissolved in DMSO (3.5 mL) and (R)-(−)-2-Amino-1-pentanol (66 mg, 0.634 mmol) and pyridine (52 μL, 0.634 mmol) was added. The reaction was heated in microwave to 170° C. for 900 sec (Parameters: 30 seconds pre-stirring, holding time on, normal absorption). The mixture was diluted with EtOAc and washed with aqueous solution of NH₄Ac. The water phase was washed with EtOAc and the organic phases were pooled, dried with MgSO₄, filtered and evaporated in vacuo. The crude product was purified on a silica column with 5:1 n-heptane:EtOAc as the mobile phase. This gave 2.1 mg (1.5%) of [4-(R)-1-hydroxymethyl-butylamino)-2-trifluoromethyl-phenyl]-acetonitrile. M/Z=286

### Example 24

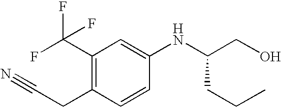

### [4-(S)-1-Hydroxymethyl-butylamino)-2-trifluoromethyl-phenyl]-acetonitrile

(4-Fluoro-2-trifluoromethyl-phenyl)-acetonitrile (100 mg, 0.492 mmol) was coupled with (S)-(+)-2-Amino-1-pentanol (66 mg, 0.634 mmol), Pyridine (52 μL, 0.634 mmol), in DMSO (3.5 mL) using the same procedure as described in Example-23. This gave 2.2 mg (1.6%) of [4-(S)-1-hydroxymethyl-butylamino)-2-trifluoromethyl-phenyl]-acetonitrile. M/Z=286

### Example 25

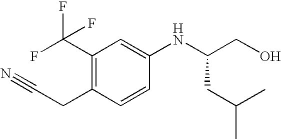

### [4-(S)-1-Hydroxymethyl-3-methyl-butylamino)-2-trifluoromethyl-phenyl]-acetonitrile

(4-Fluoro-2-trifluoromethyl-phenyl)-acetonitrile (119 mg, 0.584 mmol) was coupled with L-Leucinol (89 mg, 0.759 mmol), Pyridine (62 μL, 0.759 mmol), DMSO (3.2 mL) using the same procedure as described in Example-23. This gave 2.6 mg (1.5%) of [4-((S)-1-hydroxymethyl-3-methyl-butylamino)-2-trifluoromethyl-phenyl]-acetonitrile. M/Z=300

### Example 26

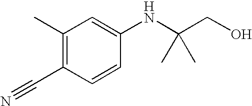

### 4-(2-Hydroxy-1,1-dimethyl-ethylamino)-2-methyl-benzonitrile

The 2-methyl-2-(3-hydroxy-4-nitro-phenylamino)-propan-1-ol (360 mg, 1.6 mmol) was dissolved in ethanol (26 ml) and Na₂S₂O₄ (2.23 g, 12.8 mmol) was added and the solution heated at 80° C. overnight. The solvent was evaporated and the remaining solid was partitioned between 10% aqueous solution NaHCO₃ and EtOAc. The water phase (pH=neutral) was extracted with EtOAc (x 3), the collected organic phase washed with brine and dried (MgSO₄). The 2-(4-amino-3-methyl-phenylamino)-2-methyl-propan-1-ol was used in the next step without further purification. (The amine oxidises on the TLC plate; brown spots upon standing.)

Sodium nitrite (NaNO₂) (190 mg, 2.75 mmol) in water (2.5 ml) was added to a solution of amine (500 mg, 2.5 mmol conc. HCl/ice (2.5 ml/2.5 g) during 5 min. followed by neutralisation by addition of solid CaCO₃. KCN (391 mg, 6 mmol) and CuCN (269 mg, 3.0 mmol) in water (1 ml) was heated at 60° C. (oil bath) and the cold, neutral diazonium salt solution was added drop wise over 15 min. Gas evolution was observed and the resulting suspension turned bright and strong orange. The reaction mixture was heated at 110° C. for 30 min, cooled, diluted with water and EtOAc and filtered through celite. The water phase was extracted with EtOAc and the collected organic phase washed with brine and dried (MgSO₄). The crude product (491 mg) was purified by flash chromatography (Hexane; Hex/EtOAc; 7:3→1.1) giving the reduced compound 2-methyl-2-(3-hydroxy-phenylamino)-propan-1-ol (93 mg) and 4-(2-hydroxy-1,1-dimethyl-ethylamino)-2-methyl-benzonitrile (108 mg, 21%) as a pale yellow solid. M/Z=204.

### Example 27

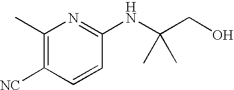

### 6-(2-Hydroxy-1,1-dimethyl-ethylamino)-2-methyl-nicotinonitrile

2-Methyl-2-(6-methyl-5-nitro-pyridin-2-ylamino)-propan-1-ol (1.08 g, 4.8 mmol) was dissolved in 75% aqueous ethanol and Na₂S₂O₄ (3.9 g, 24 mmol) was added in portions. The reaction mixture was heated at 60° C. for 30 min when TLC (10% MeOH in DCM) showed full conversion. The heat was turned off, the reaction mixture stirred overnight at ambient temperature and evaporated to dryness. To this residue was added NaHCO₃ (5% aq.) and EtOAc, the phases separated and the water phase (pH 7-8) extracted extensively with EtOAc. (The product is very water-soluble and it is probably better to do a continous extraction with EtOAc to get a higher yield). The collected organic phase was washed with brine before drying (MgSO₄). Upon standing, the colour of the organic solution turned from yellow to orange. Filtration and evaporation yielded 0.648 g (69%) of amine as a red oil.

NaNO₂ (0.25 g, 3.65 mmol) in water (3 ml) was added to a solution of amine 6 (0.648 g, 3.3 mmol) in ice/conc. HCl (3.5 g/3.5 ml) during 5 min. followed by neutralisation by addition of solid CaCO₃. KCN (0.52 g, 7.96 mmol) and CuCN (0.36 g, 3.98 mmol) in water (3 ml) was heated at 60° C. (oil bath) and the cold, neutral diazonium salt solution was added drop wise over 15 min. Gas evolution was observed and the resulting suspension turned bright and strong orange. The reaction mixture was heated at 110° C. for 30 min, cooled, diluted with water and EtOAc and filtered through celite. The water phase was extracted with EtOAc and the collected organic phase was washed with brine and dried (MgSO₄). The crude product (0.248 g) was purified by flash chromatography (Hexane→Hex:EtOAc 3:7) yielding 34 mg of 2-methyl-2-(6-methyl-pyridin-2-ylamino)-propan-1-ol and 11 mg of 6-(2-hydroxy-1,1-dimethyl-ethylamino)-2-methyl-nicotinonitrile. M/Z=205.

### Example 28

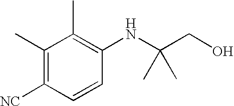

### 4-(2-Hydroxy-1,1-dimethyl-ethylamino)-2,3-dimethyl-benzonitrile

The nitro compound 18 (0.20 g, 0.84 mmol) was dissolved in EtOH (20 ml), Na₂S₂O₄ (1.1. g, 6.71 mmol) was added and the reaction mixture heated at 80° C. overnight. The cold reaction mixture was filtered through celite, washed well with EtOAc and the filtrate evaporated to dryness. The crude 2-(4-amino-1,3-dimethyl-phenylamino)-2-methyl-propan-1-ol (0.292 g), pure by ¹H-NMR, was used as such in the next reactions.

The reaction was performed using the same procedure as described in Example-21 using 2-(4-amino-2,3-dimethyl-phenylamino)-2-methyl-propan-1-ol (0.175 g, 0.84 mmol) in conc. HCl/ice water (1 ml/5 ml), NaNO₂ (64 mg mg, 0.92 mmol) in water (1 ml), KCN (130 mg, 2 mmol) and CuCN (90 mg, 1 mmol) in water (1 ml). The crude product (341 mg) was purified by flash chromatography (Hexane; Hex 7/EtOAc 3) giving reduced compound 2-(2,3-dimethyl-4-nitro-phenylamino)-2-methyl-propan-1-ol and 4-(2-hydroxy-1,1-dimethyl-ethylamino)-2,3-dimethyl-benzonitrile. All the fractions containing impure nitrile were collected and crystallised from hexane/EtOAc to give 25 mg (13%) of pure 4-(2-hydroxy-1,1-dimethyl-ethylamino)-2,3-dimethyl-benzonitrile. M/Z=218.

### Procedure for Library Synthesis (Examples 29-86)

The following is the general procedure for library synthesis for the examples of 29-88. The compounds are shown in table 2.

Smith-vials for the microwave oven were charged with 0.1 mmol either of the starting materials; 5-fluoro-2-nitro toluene, 5-fluoro-2-nitrobenzotrifluoride, 6-fluoro-2-methyl-3-nitro-pyridine.

To each vial was added 0.5 ml DMSO, 20 μL triethylamine (1.4 equivalents), and 1.4 equivalents of the diverse amino alcohols. The vials were run 1100 s in 140° C. in a microwave oven. After synthesis the products were analysed by LC-MS. The DMSO solutions were transferred to test tubes, and evaporated onto silica gel under reduced pressure. The silica gel from the tubes was placed on SPE SI columns, and a frit was placed on top. The products were purified with a gradient solution of heptane/EtOAc. The fractions were pooled and solvent was evaporated. Compounds which were more than 90% pure were tested in an in vitro assay which is described below. Purity was determined by analytic HPLC.

The scaffold used for the construction of the library is according to Formula II. The

| | | | | | Formula II |
|---|---|---|---|---|---|
| 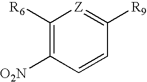 | | | | | |
| Example | R9 | R6 | Z | Yield (%) | MS (-Q1) |
| 29 | 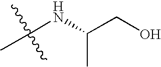 | CF₃ | CH | 46 | 262.9 |
| 30 | 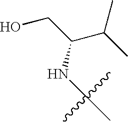 | CF₃ | CH | 55 | 290.8 |
| 31 | 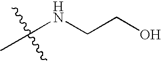 | CF₃ | CH | 24 | 249.1 |
| 32 | 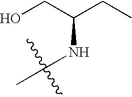 | CF₃ | CH | 62 | 276.7 |
| 33 | 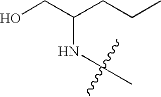 | CF₃ | CH | 65 | 290.8 |
| 34 | 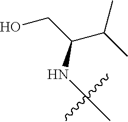 | CF₃ | CH | 23 | 290.8 |
| 35 | 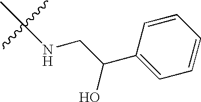 | CF₃ | CH | 93 | 325.3 |
| 36 | 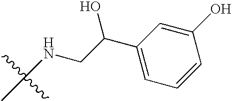 | CF₃ | CH | 78 | 341.2 |
| 37 | 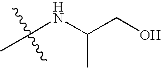 | CF₃ | CH | 82 | 262.9 |
| 38 | 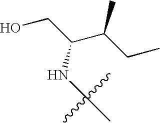 | CF₃ | CH | 95 | 305.2 |
| 39 | 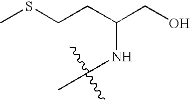 | CF₃ | CH | 98 | 323.2 |
| 40 | 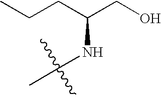 | CF₃ | CH | 98 | 290.8 |
| 41 | 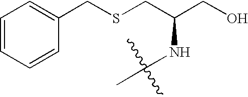 | CF₃ | CH | 89 | 385 |
| 42 | 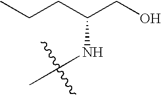 | CF₃ | CH | 92 | 290.8 |
| 43 | 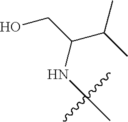 | CF₃ | CH | 95 | 290.8 |
| 44 | 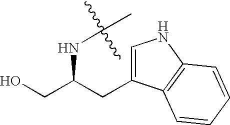 | CF₃ | CH | 100 | 378.1 |
| 45 | 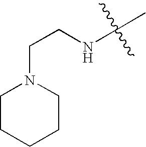 | CF₃ | CH | 84 | 316 |
| 47 | 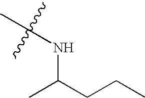 | CF₃ | CH | 106 | 275.2 |
| 48 | 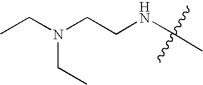 | CF₃ | CH | 75 | 304.3 |
| 50 | 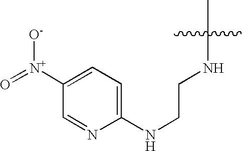 | CF₃ | CH | 76 | 370 |
| 52 | 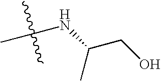 | CH₃ | N | 53 | 238.0 |
| 53 | 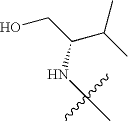 | CH₃ | N | 53 | 238.0 |
| 54 | 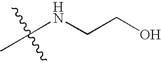 | CH₃ | N | 30 | 195.7 |
| 55 | 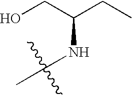 | CH₃ | N | 60 | 223.9 |
| 56 | 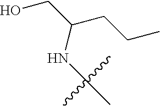 | CH₃ | N | 63 | 238.0 |
| 57 | 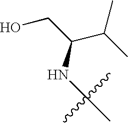 | CH₃ | N | 22 | 238.0 |
| 58 | 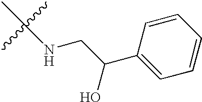 | CH₃ | N | 88 | 272.2 |
| 59 | 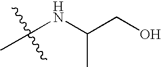 | CH₃ | N | 65 | 209.8 |
| 60 | 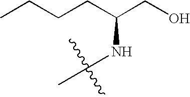 | CH₃ | N | 60 | 252.1 |
| 61 | 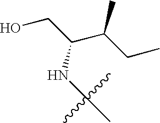 | CH₃ | N | 79 | 252.1 |
| 62 | 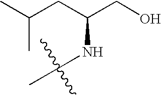 | CH₃ | N | 89 | 252.1 |
| 63 | 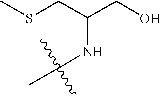 | CH₃ | N | 74 | 270.4 |
| 64 | 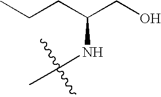 | CH₃ | N | 84 | 238.0 |
| 65 | 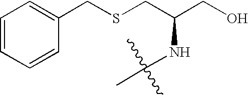 | CH₃ | N | 78 | 332.2 |
| 66 | 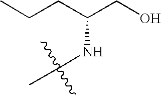 | CH₃ | N | 88 | 238.0 |
| 67 | 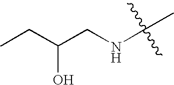 | CH₃ | N | 80 | 224.2 |
| 68 | 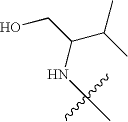 | CH₃ | N | 75 | 238.0 |
| 73 | 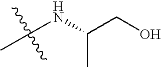 | CH₃ | C | 44.0 | 208.9 |
| 74 | 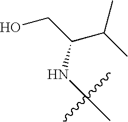 | CH₃ | CH | 55.0 | 237.1 |
| 75 | 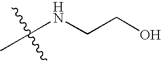 | CH₃ | CH | 66.0 | 195.1 |
| 76 | 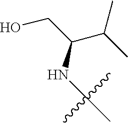 | CH₃ | CH | 31.0 | 237.1 |
| 77 | 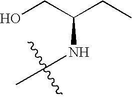 | CH₃ | CH | 30.0 | 223 |
| 78 | 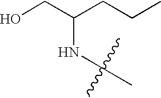 | CH₃ | CH | 32.0 | 237.1 |
| 79 | 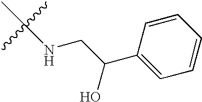 | CH₃ | CH | 27 | 271.3 |
| 80 | 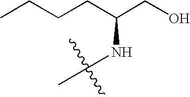 | CH₃ | CH | 25 | 250.9 |
| 81 | 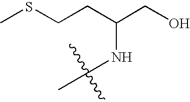 | CH₃ | CH | 27 | 269.2 |
| 82 | 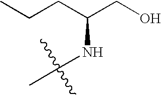 | CH₃ | CH | 24 | 237.1 |
| 83 | 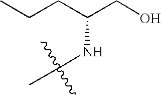 | CH₃ | CH | 24 | 237.1 |
| 84 | 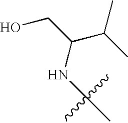 | CH₃ | CH | 24 | 237.1 |
| 86 | 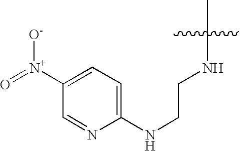 | CH₃ | CH | 33 | 316 |

### Example 87

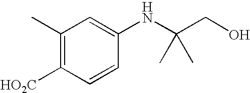

### 4-(2-Hydroxy-1,1-dimethyl-ethylamino)-2-methyl-benzoic acid

A suspension of 4-(2-hydroxy-1,1-dimethyl-ethylamino)-2-methyl-benzonitrile (70 mg, 0.34 mmol) and NaOH (0.14 g, 3.4 mmol) in water/MeOH (5 ml/8ml) was refluxed for 4 days. The reaction mixture was diluted with water, pH adjusted to approx. 3 with 50% aq. HCl. The precipitated solid was filtered off and collected, the water phase was extracted with EtOAc (x 3), washed with brine and dried (MgSO₄). The crude product was purified on a silica column with 1:1 n-heptane:EtOAc as mobile phase. This gave 39 mg (51%) of the 4-(2-hydroxy-1,1-dimethyl-ethylamino)-2-methyl-benzoic acid as a brownish foam. M/Z 223.

### Example-88

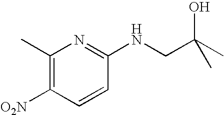

### 2-Methyl-N-(6-methyl-5-nitro-pyridin-2-yl amino)-propan-2-ol

2-(6-Methyl-5-nitro-pyridin-2-ylamino)-butionic methyl ester (30 mg, 0.13 mmol) was dissolved in THF (3 mL) and added to a nitrogen-pursed flask containing methyl magnesium chloride (MeMgCl) (0.08 ml, 0.0.27 mmol)at 0° C. The reaction mixture was allowed to reach room temperature and then refluxed for 5 h. The reaction was quenched by adding saturated NH₄Cl. The reaction mixture was extracted with EtOAc and washed with H₂O and brine. The crude product was purified by HPLC. This gave 1.5 mg (5%) of 2-methyl-N-(6-methyl-5-nitro-pyridin-2-yl amino)-propan-2-ol as yellow oil. M/Z=225.

### Example 89

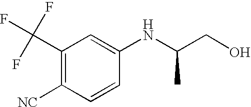

### 4-((R)-2-Hydroxy-1-methyl-ethylamino)-2-trifluoromethyl-benzonitrile

D-Alanine (36 mg, 0.40 mmol) was dissolved in THF (dry, 1 ml) and the vials were purged with N₂ for 5 min. BF₃-Et₂O (0.050 ml 0.40 mmol) was added with syringe and the mixture was heated at 70° C. for 1.5 h. BH₃—SMe₂ (0.22 ml, 0.44 mmol, 2M solution) was added carefully during vigorous stirring (an exoterm was formed approx half way) (a evolution of gas was noticed). The reactions was purged with N₂ and then heated at 70° C. over night (17 h). The reaction was allowed to cool to room temp. The excess borane was quenched by addition of 1 ml of a 1:1 mixture of THF:H₂O, followed by 1 ml of NaOH (5M). The two phase system was heated at 70° C. in 4 h. The flask was purged with N₂ to blow off the THF. CH₂Cl₂ (2 ml) was added and the two phase system was transformed to a Phase separator. Additional CH₂Cl₂ (2 ml) was added and the combined organic phases were evaporated. The crude (21 mg) was then dissolved in DMSO and the reaction was continued as in example 1. 4-Fluoro-2-trifluoromethyl-benzonitrile (19 mg, 0.1 mmol) was coupled with the formed (R)-2-amino-propan-1-ol. DIPEA (0.021 ml, 0.12 mmol), in 1 mL DMSO using the same procedure as described in Example-1. Purification on preperative HPLC gave 4 mg (16%) of 4-((R)-2-Hydroxy-1-methyl-ethylamino)-2-trifluoromethyl-benzonitrile as a white solid. M/Z=244.

### Example 90

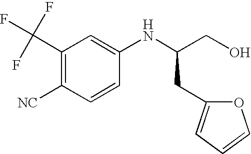

### 4-((R)-1-Furan-2-ylmethyl-2-hydroxy-ethylamino)-2-trifluoromethyl-benzonitrile

(R)-2-Amino-3-furan-2-yl-propionic acid (40 mg, 0.25 mmol) was reduced using the same procedure as described in Example-90. The crude was coupled with 4-Fluoro-2-trifluoromethyl-benzonitrile (19 mg, 0.1 mmol) and DIPEA (0.05 ml, 0.2 mmol) as in example 1 and gave 4-(R)-1-furan-2-ylmethyl-2-hydroxy-ethylamino)-2-trifluoromethyl-benzonitrile 11 mg (29%), after purification on HPLC, as a white solid. M/Z=310.

### Example 91

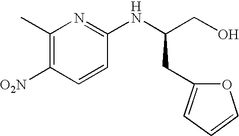

### (R)-3-Furan-2-yl-2-(6-methyl-5-nitro-pyridin-2-ylamino)-propan-1-ol

(R)-2-Amino-3-furan-2-yl-propionic acid (40 mg, 0.25 mmol) was reduced using the same procedure as described in Example-90. The crude was coupled with 6-chloro-3-nitro-2-picoline (17 mg, 0.1 mmol) and DIPEA (0.05 ml, 0.2 mmol) as in example 1 and gave, after purification on HPLC, 9 mg (33%) of (R)-3-Furan-2-yl-2-(6-methyl-5-nitro-pyridin-2-ylamino)-propan-1-ol, as a white solid. M/Z=277.

### Example 92

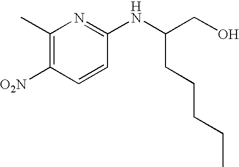

### 2-(6-Methyl-5-nitro-pyridin-2-ylamino)-heptan-1-ol

2-Amino-heptanoic acid (33 mg, 0.25 mmol) was reduced using the same procedure as described in Example-90. The crude was coupled with 6-chloro-3-nitro-2-picoline (17 mg, 0.1 mmol) and DIPEA (0.05 ml, 0.2 mmol) as in Example 1 and gave after purification on HPLC, 3 mg (11%) 2-(6-methyl-5-nitro-pyridin-2-ylamino)-heptan-1-ol, as an oil. M/Z=267

### Example 93

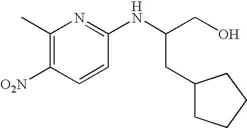

### 3-Cyclopentyl-2-(6-methyl-5-nitro-pyridin-2-ylamino)-propan-1-ol

2-Amino-3-cyclopentyl-propionic acid (36 mg, 0.25 mmol) was reduced using the same procedure as described in Example-90. The crude was coupled with 6-chloro-3-nitro-2-picoline (17 mg, 0.1 mmol) and DIPEA (0.05 ml, 0.2 mmol) as in Example 1 and gave, after purification on HPLC, 4 mg (14%) 3-Cyclopentyl-2-(6-methyl-5-nitro-pyridin-2-ylamino)-propan-1-ol, as an yellow solid. M/Z=279.

### Example 94

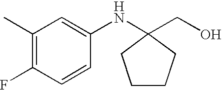

### [1-(4-Fluoro-3-methyl-phenylamino)-cyclopentyl]-methanol

4-Fluoro-2-methyl phenol (0.24 mmol) was solved in 800 μL DMSO. (1-Amino-cyclopentyl)-methanol (0.29 mmol) was added and then Diisopropyl-ethyl amine (DIPEA) (0.29 mmol). Reaction was heated to 180° C. in microwave for 15 min (Parameters: Normal absorption, hold time on, pre-stirring 20 sec). Starting material was remaining so reaction was heated to 220° C. for additional 15 min. Several products obtained. Crude mixture was diluted in CH₂Cl₂ and washed several times with NH₄Cl (aq) and phases were separated on SPE Phase Separator. Organic phase was evaporated in vacuo and crude product mixture was then purified on silica column with 5:1 n-heptane:EtOAc as mobile phase. This gave 2.3 mg (4%) of [1-(4-fluoro-3-methyl-phenylamino)-cyclopentyl]-methanol. M/Z=221

### Example 95

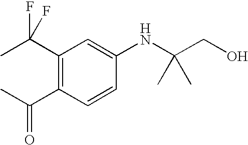

### 1-[4-(2-Hydroxy-1,1-dimethyl-ethylamino)-2-trifluoromethyl-phenyl]-ethanone

1-(4-Fluoro-2-trifluoromethyl-phenyl)-ethanone (40mg, 0.194 mmol) was solved in 800 μL DMSO. 2-Amino-2-methyl-propan-1-ol (23 mg, 0.252 mmol) was added and then DIPEA (44 μL, 0.252 mmol). Reaction mixture was heated to 180° C. in microwave for 15 min (Parameters: Normal absorption, hold time on, pre-stirring 25 sec). Majority of starting material still left so reheated to 210° C. for 15 min. Several products obtained. Crude mixture was diluted in CH₂Cl₂ and washed several times with NH₄Cl (aq) and phases were separated on SPE Phase Separator. Organic phase was evaporated in vacuo and crude product mixture was then purified on silica column with 10:1 n-heptane:EtOAc as mobile phase. This gave 3 mg (6%) of 1-[4-(2-Hydroxy-1,1-dimethyl-ethylamino)-2-trifluoromethyl-phenyl]-ethanone as minor product. M/Z=275.

### Example 96

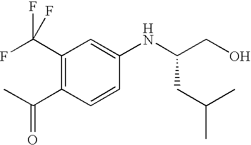

### 1-[4-((S)-1-Hydroxymethyl-3-methyl-butylamino)-2-trifluoromethyl-phenyl]-ethanone

1-(4-Fluoro-2-trifluoromethyl-phenyl)-ethanone (40 mg, 0.194 mmol) was coupled with (S)-2-Amino-4-methyl-pentan-1-ol (30 mg, 0.252 mmol), DIPEA (44 μL, 0.252 mmol) in DMSO 800 μL using the same procedure as described in Example-97. This gave 15 mg (25%) of 1-[4-((S)-1-hydroxymethyl-3-methyl-butylamino)-2-trifluoromethyl-phenyl]-ethanone. M/Z=303

### Example 97

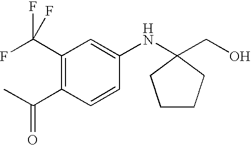

### 1-[4-(1-Hydroxymethyl-cyclopentylamino)-2-trifluoromethyl-phenyl]-ethanone

1-(4-Fluoro-2-trifluoromethyl-phenyl]-ethanone (40 mg, 0.194 mmol) was coupled with (1-Amino-cyclopentyl)-methanol (29 mg, 0.252 mmol), DIPEA (44 μL, 0.252 mmol), in DMSO 800 μL using the same procedure as described in Example-97. This gave 5 mg (9%) of 1-[4-(1-hydroxymethyl-cyclopentylamino)-2-trifluoromethyl-phenyl]-ethanone. M/Z=301.

### Example 98

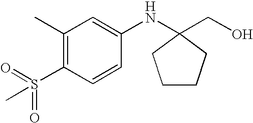

### [1-(4-Methanesulfonyl-3-methyl-phenylamino)-cyclopentyl]-methanol

4-Fluoro-1-methanesulfonyl-2-methyl-benzene (40 mg, 0.213 mmol) was solved in 800 μL DMSO. (1-Amino-cyclopentyl)-methanol (32 mg, 0.276 mmol) was added and DIPEA (48 μL, 0.276 mmol). Reaction mixture was heated to 180° C. in microwave for 15 min (Parameters: Normal absorption, hold time on, pre-stirring 30 sec). Crude mixture was diluted in CH₂Cl₂ and washed several times with NH₄Cl (aq) and phases were separated on SPE Phase Separator. Organic phase was evaporated in vacuo and crude product mixture was then purified on silica column with 7:1 n-heptane:EtOAc as mobile phase. This gave 1.4 mg (2%) of [1-(4-methanesulfonyl-3-methyl-phenylamino)-cyclopentyl]-methanol. M/Z=283

### Example 99

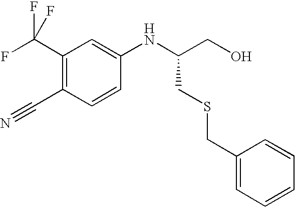

### 4-((R)-1-Benzylsulfanylmethyl-2-hydroxy-ethylamino)-2-trifluoromethyl-benzonitrile

4-Fluoro-2-trifluoromethyl-benzonitrile (60 mg, 0.32 mmol) was solved in 1000 μL DMSO. (R)-2-amino-3-benzylsulfanyl-propan-1-ol (81 mg, 0.41 mmol) was added and then diisopropyl-ethyl amine (DIPEA) (53 mg, 0.41 mmol). Reaction was heated to 180° C. in microwave for 15 min (Parameters: Normal absorption, hold time on, pre-stirring 20 sec). Crude mixture was diluted with CH₂Cl₂ and washed several times with NH₄Cl (aq) and phases were separated on SPE Phase Separator. Organic phase was evaporated in vacuo and crude product mixture was then purified on silica column with 3:1 n-heptane:EtOAc as mobile phase. This gave pure product 82 mg (71%) of 4-((R)-1-benzylsulfanylmethyl-2-hydroxy-ethylamino)-2-trifluoromethyl-benzonitrile as transparent oil. M/Z=366

### Example 100

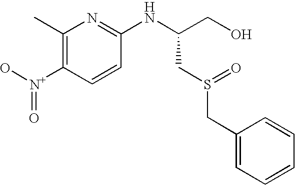

### (R)-2-(6-Methyl-5-nitro-pyridin-2-ylamino)-3-phenylmethanesulfinyl-propan-1-ol

CH₂Cl₂ (0.125 mL) was cooled to 0° C. and mCPBA (13 mg, 0.07 mmol) was solved in it. Stirred at 0° C. for 10 min then (R)-3-benzylsulfanyl-2-(6-methyl-5-nitro-pyridin-2-ylamino)-propan-1-ol (20 mg, 0.06 mmol) was added. Stirred at 0° C. for 20 min. Cooling bath was removed and reaction was allowed to warm to room temperature and was then stirred overnight. The organic phase was washed with brine, phases were separated on SPE Phase Separator and organic phase was dried and evaporated in vacuo. Crude product gives precipitation on salvation in 3:1 n-Heptane:EtOAc. Precipitate was consisting of mainly product and was solved in acetonitrile and purified on silica column with EtOAc as mobilephase. This gave 8.2 mg (39%) of (R)-2-(6-Methyl-5-nitro-pyridin-2-ylamino)-3-phenylmethanesulfinyl-propan-1-ol. M/Z=349.

### Example 101

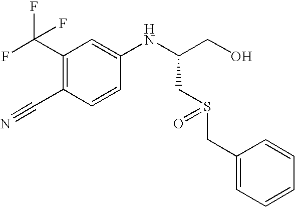

### 4-((R)-2-Hydroxy-1-phenylmethanesulfinylmethyl-ethylamino)-2-trifluoromethyl-benzonitrile

4-((R)-1-Benzylsulfanylmethyl-2-hydroxy-ethylamino)-2-trifluoromethyl-benzonitrile (20 mg, 0.06 mmol) was reacted with mCPBA (11 mg, 0.07 mmol) in CH₂Cl₂ (0.125 mL) using the same procedure as described in Example-104. This gave 14.1 mg (67%) of 6-((R)-2-Hydroxy-1-phenylmethanesulfinylmethyl-ethylamino)-2-trifluoromethyl-nicotinonitrile. M/Z—382

### Example 102

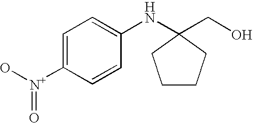

### [1-(4-Nitro-phenylamino)-cyclopentyl]-methanol

1-Fluoro-4-nitro-benzene (41 mg, 0.29 mmol) was solved in 1000 μL DMSO. (1-amino-cyclopentyl)-methanol (44 mg, 0.38 mmol) was added and then diisopropyl-ethyl amine (DIPEA) (49 mg, 0.38 mmol). Reaction was heated to 170° C. in microwave for 15 min (Parameters: Normal absorption, hold time on, pre-stirring 30 sec). Crude mixture was diluted in EtOAc and washed several times with NH₄Cl (aq) and phases were separated. Organic phase was dried and then evaporated in vacuo. Crude product mixture was purified on silica column with 3:1 n-heptane:EtOAc as mobile phase. This gave 48 mg (70%) of [1-(4-nitro-phenylamino)-cyclopentyl]-methanol. M/Z=236.

### Example 103

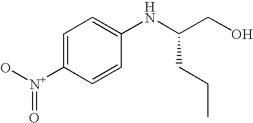

### (S)-2-(4-Nitro-phenylamino)-pentan-1-ol

1-Fluoro-4-nitro-benzene (41 mg, 0.29 mmol) was coupled with (S)-2-amino-pentan-1-ol (39 mg, 0.38 mmol), diisopropyl-ethyl amine (DIPEA) (49 mg, 0.38 mmol) in DMSO 1000 μL using the same procedure as described in Example-106. This gave 53 mg (81%) of (S)-2-(4-nitro-phenylamino)-pentan-1-ol. M/Z=224

### Example 104

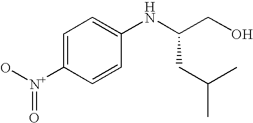

### (S)-4-Methyl-2-(4-nitro-phenylamino)-pentan-1-ol

1-Fluoro-4-nitro-benzene (42 mg, 0.29 mmol) was coupled with (S)-2-amino-4-methyl-pentan-1-ol (50 mg, 0.38 mmol), diisopropyl-ethyl amine (DIPEA) (50 mg, 0.38 mmol) in DMSO 1000 μL using the same procedure as described in Example-106. This gave 40 mg (57%) of (S)-4-Methyl-2-(4-nitro-phenylamino)-pentan-1-ol. M/Z=238.

### Example 105

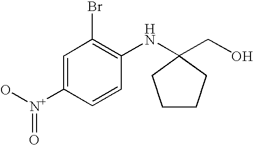

### [1-(2-Bromo-4-nitro-phenylamino)-cyclopentyl]-methanol

[1-(4-Nitro-phenylamino)-cyclopentyl]-methanol (10 mg, 0.042 mmol) was solved in a 1:1 mixture of CH₂Cl₂:MeOH (2 mL). CaCO₃ (8.5 mg, 0.085 mmol) was added and the solution was stirred at room temp for 10 min. Benzyltrimethylammonium tribromide (36 mg, 0.093 mmol) was added and the reaction was stirred at room temp for 48 h. Crude reaction was diluted with CH₂Cl₂ and washed with NH₄Cl_{(aq)} Organic phase was collected, dried and evaporated in vacuo. Crude product was purified on silica column. This gave 11 mg (83%) of [1-(2-bromo-4-nitro-phenylamino)-cyclopentyl]-methanol. M/Z=315.

### Example 106

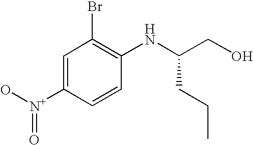

### (S)-2-(2-Bromo-4-nitro-phenylamino)-pentan-1-ol

(S)-2-(4-Nitro-phenylamino)-pentan-1-ol (29 mg, 0.13 mmol) was treated benzyltrimethylammonium tribromide (111 mg, 0.29 mmol) and CaCO₃ (26 mg, 0.26 mmol) in 1:1 mixture of CH₂Cl₂:MeOH (2 mL) using the same procedure as described in Example-109. This gave 16 mg (41%) of (S)-2-(2-Bromo-4-nitro-phenylamino)-pentan-1-ol. M/Z=303.

### Example 107

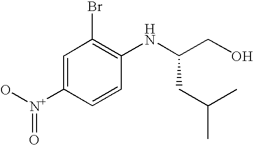

### (S)-2-(2-Bromo-4-nitro-phenylamino)-4-methyl-pentan-1-ol

(S)-4-Methyl-2-(4-nitro-phenylamino)-pentan-1-ol (29 mg, 0.13 mmol) was treated benzyltrimethylammonium tribromide (111 mg, 0.30 mmol) and CaCO₃ (26 mg, 0.27 mmol) in 1:1 mixture of CH₂Cl₂:MeOH (2 mL) using the same procedure as described in Example-109. This gave 20 mg (47%) of (S)-2-(2-Bromo-4-nitro-phenylamino)-4-methyl-pentan-1-ol. M/Z=317.

All molecules were named by Autonom 2000, part of was IS/Draw 2.5

All naming done by was IS/Draw 2.5 with Autonom 2000

### Example-108

### AR Competition Binding Assay

Recombinant human androgen receptor (hAR) was extracted from Sf9 insect cells with buffer containing 1 mM EDTA, 20 mM K₂HPO₄, 8.7% glycerol, 20 mM Na₂MoO₄ and 12 mM MTG at 5*10⁷ cells/ml. The cell debris was removed by centrifugation and the supernatant aliquoted and stored at −70° C.

An aliquot of AR extract was thawed on ice prior to use and diluted to approximately 0.2 nM (1 to 30 dilution) in buffer (100 mM KₙHₘPO₄ pH 7.0, 1 mM EDTA, 8.7% glycerol, 20 mM Na₂MoO₄ and 1 mM DTT). The test ligands were diluted in DMSO as a dilution series of 10 concentrations in duplicate, with 1:5 dilution between each concentration. Tritiated mibolerone (³H-Mib) was used as tracer compound and diluted to 1.6 nM in 1 mM EDTA, 20 mM Na₂MoO₄, 8.7% glycerol and 1 mM DTT. To a 96-well polypropylene-plate 110 μl/well of 1.6 nM ³H-Mib, 10 μl/well test substance and 110 μl/well diluted AR was added. The plates were covered and incubated at +4° C. over night. The plates were harvested on filters to separate bound ligand from unbound ligand with a Tomtec Harvester. A prewet buffer containing 20 mM Kₙ(PO₄) pH 7.6, 1 mM EDTA, v/v 0.5% polyethyleneimine was used to equilibrate the filter before filtering the samples and washing the filters with 20 mM Kₙ(PO₄) pH 7.6, 1 mM EDTA 8 times. The filters were allowed to dry for 1 hour at +65° C. A scintillating wax was melted upon the filter and the radioactivity retained on the filter was measured in a Wallac Microbeta scintillation counter.

The affinity to AR was evaluated by a non-linear four-parameter logisitic model: b=(bmax−bmin)/(1+(IC50/I)̂S)+bmin, where bmax=total concentration of binding sites, bmin=non-specific binding, I=added concentration of binding inhibitor, IC50=concentration of binding inhibitor at half-maximal binding and S=slope factor. Table: Antagonist and partial antagonist and binding activity of androgen receptor modulator compounds.

### AR Transactivation Assays

The agonist and antagonist properties of compounds were determined using a cell-based system expressing stably integrated androgen receptor and an androgen responsive reporter gene. CV-1 cells (kidney fibroblasts) stably expressing CMV-hAR and alkaline phosphatase (ALP) driven by an MMTV promoter containing an androgen response element were cultured in Dulbecco's Modified Eagle Medium (DMEM), low glucose supplemented with 10% fetal bovin serum, 1% L-glutamine, and 0.7% Hygromycine B. The stably integrated cells (ARAF) were trypsinized and resuspended in Opti-MEM 1 supplemented with 2% fetal bovine serum, 1% L-Glutamine, 50 μg/ml Gentamicine and 1% Pen/Strep. The cells were counted in a Birch chamber and diluted to a concentration of 100 000 cells/ml. The cells were then seeded out in 384 plates, 5000 cells/well in 50 μl seeding media and incubated overnight in 37 C, 5% CO₂.

The next day, the seeding medium was removed from the cells and 20 μl induction media (Opti-MEM 1 supplemented with 1% L-Glutamine, 50 μg/ml Gentamicine and 1% Pen/Strep)+/−0.1 nM Mibolerone was added to the wells. 10 μl of test compound diluted in induction media was then added to the wells. The cells were incubated 48 hr in 37 C, 5% CO₂.

After 48 hr 5 μl of cell medium was added to white 384 plates with 100 μl of ALP substrate buffer. The plates were incubated in 37 C for 20 minutes followed by incubation at room temperature for 10 minutes before each well was read in a μBETA machine. Agonist activity was calculated from the alkaline phosphatase activity induced in the absence of Mibolerone and compared to standard activation curve generated by Mibolerone alone. Antagonist activity was calculated from the decrease in ALP activity in the presence of 0.1 nM Mibolerone. EC50 and IC50 values were calculated by using a non-linear four-parameter fit as described above.

Other assays to determine androgen receptor mediated activity of the test compounds include modulation of endogenous AR mediated transcription in cell culture systems; modulation of androgen responsive tissue effects in rodents; identification of receptor surface conformation changes; and binding specificity to AR versus other nuclear receptors.

| | AR_LT IC50 | ARAF EC50 | ARAF % | ARAF IC50 | ARAF % |
|---|---|---|---|---|---|
| | (nM) | (nM) | AGONIST | (nM) | ANTAGONIST |
| Example-1 | 22.77 | 26.8 | 51.7 | 2.1 | 33.1 |
| Example-5 | 38.06 | 81.7 | 29.3 | 7.2 | 61 |
| Example-8 | 241.44 | 374.2 | 10.6 | 22.3 | 82.5 |
| Example-19 | 130.38 | | | 22.4 | 95.6 |
| Example-30 | 113.45 | 1069.9 | 7.3 | 68.3 | 88.7 |
| Example-41 | 65.10 | | | 490.3 | 71.7 |
| Example-42 | 485.50 | | | 493.3 | 92 |
| Example-60 | 68.30 | 336.3 | 9.3 | 27.4 | 79.3 |
| Example-61 | 89.30 | | | 68.3 | 87.4 |
| Example-65 | 6.20 | 1867.3 | 7 | 78.0 | 89.2 |
| Example-78 | 54.50 | 279.7 | 25 | 25.8 | 65.4 |
| Example-86 | 443.40 | | | 350.7 | 100 |
| Example-103 | 98.70 | | | 135.5 | 92.9 |
| Example-106 | 170.30 | | | 240.7 | 88.2 |

1.-14. (canceled)

15. A pharmaceutical composition containing a compound as defined in Formula I 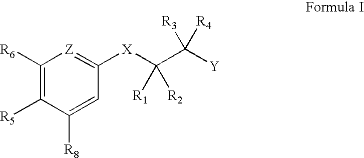
in which;
R₁ and R₂ are the same or different and independently selected from hydrogen, halogen, C₁-C₁₀ alkyl, C₁-C₁₀ substituted alkyl, C₂-C₁₀ alkenyl, C₂-C₁₀ alkynyl, C₁-C₁₀ alkoxy, C₁-C₁₀ alkenoxy, C₁-C₁₀ alkynoxy, C₁-C₁₀ alkylthio, C₁-C₁₀ alkenylthio, C₁-C₁₀ alkynylthio, C₆-C₁₀ arylthio, C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alkynylsulphoxide, C₆-C₁₀ arylsulphoxide, C₁-C₁₀ alkylarylthio, C₁-C₁₀ alkylarylsulphone, C1-C₁₀ alkylarylsulphoxide, C₆-C₁₀ aryl, or C₅-C₂₀ heteroaryl, optionally substituted with 0, 1, 2 or 3 groups of R^{a} which groups may be the same or different; or R₁ and R₂ may together form a C₃-C₁₀ cycloalkyl group;
R₃ and R₄ are the same or different and independently selected from hydrogen, halogen, C₁-C₂₀ alkyl, C₃-C₇ cycloalkyl, C₂-C₄ alkenyl, C₂-C₄ alkynyl, C₁-C₄ alkoxy, C₁-C₄ alkenoxy, C₁-C₄ alkynoxy, C₁-C₄ alkylthio, C₁-C₄ alkenylthio, C₁-C₄ alkynylthio C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alkynylsulphoxide, C₆-C₁₀ arylsulphoxide, C₁-C₁₀ alkylarylthio, C₁-C₁₀ alkylarylsulphone, C₁-C₁₀ alkylarylsulphoxide, C₆-C₁₅ aryl, C₅-C₂₀ heteroaryl optionally substituted with 0, 1, 2 or 3 groups of R^{a} which groups may be the same or different; or can together form a keto group;
R₅ is chosen from nitro, cyano, —CH₂CN, —COMe, or —SO₂CH₃;
R₆ is chosen from the group consisting of; hydrogen, C₁-C₅ alkyl, halogen, CN, CO₂H, CHF₂, CH₂F or CF₃;
Z is chosen from CR₇ or N;
R₇ is chosen from the group consisting of; H or C₁-C₅ alkyl;
R₈ is chosen from the group consisting of; hydrogen, C₁-C₅ alkyl, halogen, CHF₂, CH₂F or CF₃;
X is —NH—;
Y is chosen from hydroxy or —NH(C₁-C₁₀ heteroaryl);
R^{a} represents a member selected from: hydrogen, halogen, —CN, OH, CO₂H, CHO, NO₂, —NH₂, —NH(C₁-C₄); N(C₁-C₄)₂, —NH(C₆ aryl), —N(C₆ aryl)₂, —NH(C₅-C₁₀ heteroaryl), and —N(C₅-C₁₀ heteroaryl)₂; or a pharmaceutically acceptable salt thereof.

16.-17. (canceled)

18. A compound as defined by Formula I: 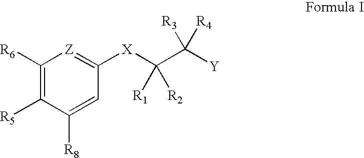
in which;
R₁ and R₂ are the same or different and independently selected from hydrogen, halogen, C₁-C₁₀ alkyl, C₁-C₁₀ substituted alkyl, C₂-C₁₀ alkenyl, C₂-C₁₀ alkynyl, C₁-C₁₀ alkoxy, C₁-C₁₀ alkenoxy, C₁-C₁₀ alkynoxy, C₁-C₁₀ alkylthio, C₁-C₁₀ alkenylthio, C₁-C₁₀ alkynylthio, C₆-C₁₀ arylthio, C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alkynylsulphone, C₆-C₁₀ arylsulphoxide, C₁-C₁₀ alkylarylthio, C₁-C₁₀ alkylarylsulphone, C₁-C₁₀ alkylarylsulphoxide, C₆-C₁₀ aryl, or C₅-C₂₀ heteroaryl, optionally substituted with 0, 1, 2 or 3 groups of R^{a} which groups may be the same or different; or R₁ and R₂ may together form a C₃-C₁₀ cycloalkyl group;
R₃ and R₄ are the same or different and independently selected from hydrogen, halogen, C₁-C₂₀ alkyl, C₃-C₇ cycloalkyl, C₂-C₄ alkenyl, C₂-C₄ alkynyl, C₁-C₄ alkoxy, C₁-C₄ alkenoxy, C₁-C₄ alkynoxy, C₁-C₄ alkylthio, C₁-C₄ alkenylthio, C₁-C₄ alkynylthio C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alkynylsulphoxide, C₆-C₁₀ arylsulphoxide, C₁-C₁₀ alkylarylthio, C₁-C₁₀ alkylarylsulphone, C₁-C₁₀ alkylarylsulphoxide, C₆-C₁₅ aryl, C₅-C₂₀ heteroaryl optionally substituted with 0, 1, 2 or 3 groups of R^{a} which groups may be the same or different; or can together form a keto group;
R₅ is chosen from nitro, cyano, —CH₂CN, —COMe, or —SO₂CH₃;
R₆ is chosen from the group consisting of; hydrogen, C₁-C₅ alkyl, halogen, CN, CO₂H, CHF₂, CH₂F or CF₃;
R₈ is chosen from the group consisting of; hydrogen, C₁-C₅ alkyl, halogen, CHF₂, CH₂F or CF₃;
X is —NH—;
Y is chosen from hydroxy, or —NH(C₁-C₁₀ heteroaryl);
Z is chosen from CR₇ or N;
R₇ is H or C₁-C₅ alkyl;
R^{a} represents a member selected from: hydrogen, halogen, —CN, OH, CO₂H, CHO, NO₂, —NH₂, —NH(C₁-C₄); N(C₁-C₄)₂, —NH(C₆aryl), —N(C₆ aryl)₂, —NH(C₅-C₁₀ heteroaryl), and —N(C₅-C₁₀ heteroaryl)₂; or a pharmaceutically acceptable salt thereof,
with the proviso that the compound is not one of: 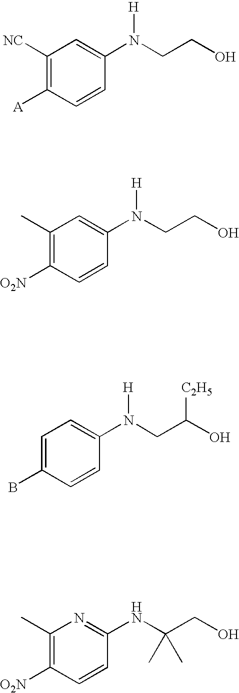
wherein A is —CN or —NO₂, and B is —CN, —NO₂ or —SO₂CH₃.

19. A compound according to claim 18, wherein R₁ or/and R₂ are H, (S)-methyl, methyl, (R)-ethyl, (S)-ethyl, ethyl, (R)-propyl, (S)-propyl, propyl, (S)-butyl, S-1-methyl-propyl, S-2-methyl-propyl, (R)-isopropyl, (S)-isopropyl, isopropyl, cyclopentyl, —(CH₂)₂SMe, (R)—CH₂SCH₂Ph, (S)-benzyl, 4-chloro-benzyl, (S)-3-methyl-1-H-indole or (S)-phenyl.

20. A compound according to claim 18, wherein R₃ is chosen from the group consisting of hydrogen, methyl, ethyl, phenyl, 3-hydroxy phenyl, 4-hydroxy phenyl, or forms a keto group together with R₄.

21. A compound according to claim 18, wherein R₄ is H, methyl, or forms a keto group together with R₃.

22. A compound according to claim 18, wherein R₅ is NO₂, CN, or CH₂CN.

23. A compound according to claim 18, wherein R₆ is Me or CF₃.

24. A compound according to claim 18, wherein R₇ is H or Me.

25. A compound according to claim 18, wherein R₈ is H or methyl.

26. (canceled)

27. A compound according to claim 18, wherein Y is —OH.

28. (canceled)

29. A compound according to claim 18, wherein the compound is chosen from the group consisting of:
2-Methyl-2-(4-nitro-3-trifluoromethyl-phenylamino)-propan-1-ol;
[1-(4-Nitro-3-trifluoromethyl-phenylamino)-cyclopentyl]-methanol
(S)-2-(4-Nitro-3-trifluoromethyl-phenylamino)-3-phenyl-propan-1-ol;
(S)-2-(4-Nitro-3-trifluoromethyl-phenylamino)-butan-1-ol;
2-Methyl-2-(3-hydroxy-4-nitro-phenylamino)-propan-1-ol;
[1-(3-Methyl-4-nitro-phenylamino)-cyclopentyl]-methanol;
(S)-2-(3-Methyl-4-nitro-phenylamino)-butan-1-ol;
2-Methyl-2-(6-methyl-5-nitro-pyridine-2-ylamino)-propan-1-ol;
[1-(6-Methyl-5-nitro-pyridine-2-ylamino)-cyclopentyl]-methanol;
(S)-2-(6-Methyl-5-nitro-pyridin-2ylamino) 2-phenyl-ethanol;
(S)-2-(6-Methyl-5-nitro-pyridine-2-ylamino)-3-phenyl-propan-1-ol;
(S)-2-(6-Methyl-5-nitro-pyridin-2-ylamino)-butan-1-ol;
(DL)-3-(4-Chloro-phenyl)-2-(6-methyl-5-nitro-pyridin-2-ylamino)-propan-1-ol;
(S)-2-(6-Methyl-5-nitro-2-pyridin-2-ylamino)-propionic acid;
(S)-2-(6-Methyl-5-nitro-pyridin-2-ylamino)-propan-1-ol;
2-(2,3-Dimethyl-4-nitro-phenylamino)-2-methyl-propan-1-ol;
(S)-2-(3,5-Dimethyl-4-nitro-phenylamino)-butan-1-ol;
4-(2-Hydroxy-1,1-dimethyl-ethylamino)-2-trifluoromethyl-benzonitrile;
4-(1-Hydroxymethyl-cyclopentylamino)-2-trifluoromethyl-benzonitrile;
(S)-4-(1-Hydroxymethyl-cyclopentylamino)-2-trifluoromethyl-benzonitrile;
(R)-4-(1-Hydroxymethyl-butylamino)-2-trifluoromethyl-benzonitrile;
(S)-4-(1-Hydroxymethyl-butylamino)-2-trifluoromethyl-benzonitrile;
[4-((S)-1-Hydroxymethyl-butylamino)-2-trifluoromethyl-phenyl]-acetonitrile;
[4-((R)-1-Hydroxymethyl-butylamino)-2-trifluoromethyl-phenyl]-acetonitrile;
[4-((S)-1-Hydroxymethyl-3-methyl-butylamino)-2-trifluoromethyl-phenyl]-acetonitrile;
4-(2-Hydroxy-1,1-dimethyl-ethylamino)-2-methyl-benzonitrile;
6-(2-Hydroxy-1,1-dimethyl-ethylamino)-2-methyl-nicotinonitrile;
4-(2-Hydroxy-1,1-dimethyl-ethylamino)-2,3-dimethyl-benzonitrile;
and compounds having the formula: 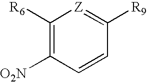
in which R₉, R₆ and Z are as defined in the following table:

| R9 | R6 | Z |
|---|---|---|
| 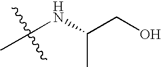 | CF₃ | CH |
| 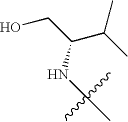 | CF₃ | CH |
| 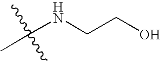 | CF₃ | CH |
| 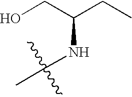 | CF₃ | CH |
| 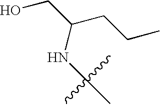 | CF₃ | CH |
| 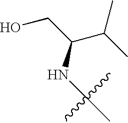 | CF₃ | CH |
| 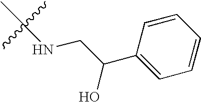 | CF₃ | CH |
| 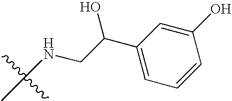 | CF₃ | CH |
| 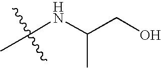 | CF₃ | CH |
| 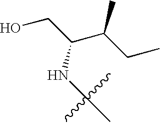 | CF₃ | CH |
| 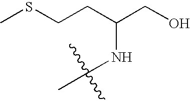 | CF₃ | CH |
| 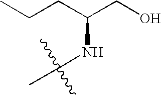 | CF₃ | CH |
| 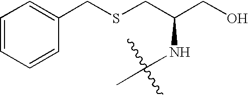 | CF₃ | CH |
| 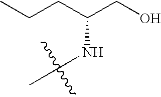 | CF₃ | CH |
| 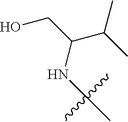 | CF₃ | CH |
| 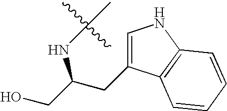 | CF₃ | CH |
| 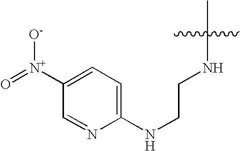 | CF₃ | CH |
| 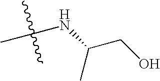 | CH₃ | N |
| 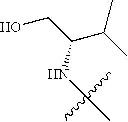 | CH₃ | N |
| 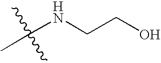 | CH₃ | N |
| 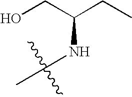 | CH₃ | N |
| 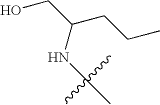 | CH₃ | N |
| 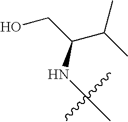 | CH₃ | N |
| 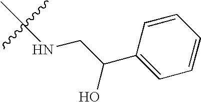 | CH₃ | N |
| 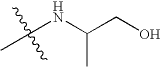 | CH₃ | N |
| 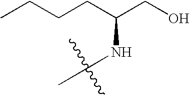 | CH₃ | N |
| 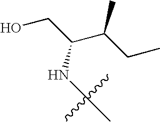 | CH₃ | N |
| 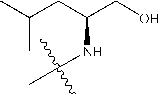 | CH₃ | N |
| 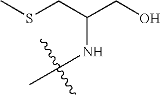 | CH₃ | N |
| 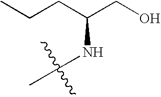 | CH₃ | N |
| 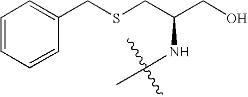 | CH₃ | N |
| 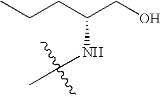 | CH₃ | N |
| 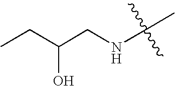 | CH₃ | N |
| 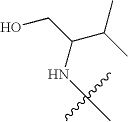 | CH₃ | N |
| 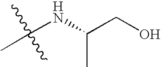 | CH₃ | CH |
| 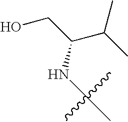 | CH₃ | CH |
| 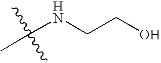 | CH₃ | CH |
| 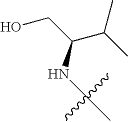 | CH₃ | CH |
| 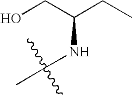 | CH₃ | CH |
| 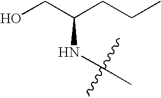 | CH₃ | CH |
| 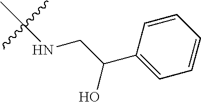 | CH₃ | CH |
| 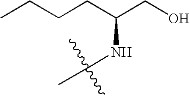 | CH₃ | CH |
| 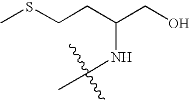 | CH₃ | CH |
| 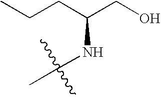 | CH₃ | CH |
| 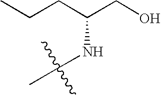 | CH₃ | CH |
| 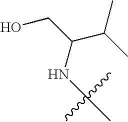 | CH₃ | CH |
| 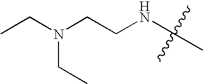 | CH₃ | CH |
| 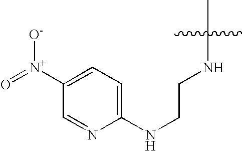 | CH₃ | CH |

2-Methyl-N-(6-methyl-5-nitro-pyridin-2-yl amino)-propan-2-ol;
4- ((R)-2-Hydroxy-1-methyl-ethylamino)-2-trifluoromethyl-benzonitrile
4-((R)-1-Furan-2-ylmethyl-2-hydroxy-ethylamino)-2-trifluoromethyl-benzonitrile
(R)-3-Furan-2-yl-2-(6-methyl-5-nitro-pyridin-2-ylamino)-propan-1-ol
2-(6-Methyl-5-nitro-pyridin-2-ylamino)-heptan-1-ol
3-Cyclopentyl-2-(6-methyl-5-nitro-pyridin-2-ylamino)-propan-1-ol
[1-(4-Methanesulfonyl-3-methyl-phenylamino)-cyclopentyl]-methanol
2,2-Dimethyl-3-(6-methyl-5-nitro-pyridin-2-ylamino)-propan-1-ol
2,2-Dimethyl-3-(3-methyl-4-nitro-phenylamino)-propan-1-ol
4-((R)-1-Benzylsulfanylmethyl-2-hydroxy-ethylamino)-2-trifluoromethyl-benzonitrile
(R)-2-(6-Methyl-5-nitro-pyridin-2-ylamino)-3-phenylmethanesulfinyl-propan-1-ol
4-((R)-2-Hydroxy-1-phenylmethanesulfinylmethyl-ethylamino)-2-trifluoromethyl-benzonitrile
[1-(4-Nitro-phenylamino)-cyclopentyl]-methanol
(S)-2-(4-Nitro-phenylamino)-pentan-1-ol
[1-(2-Bromo-4-nitro-phenylamino)-cyclopentyl]-methanol
(S)-2-(2-Bromo-4-nitro-phenylamino)-pentan-1-ol
(S)-2-(2-Bromo-4-nitro-phenylamino)-4-methyl-pentan-1-ol

30. A compound according to claim 18, wherein R₁ or R₂ is a C₆-C₁₀ arythio comprising an aryl-substituted sulfur-containing C₁-C₁₀ alkyl group.

31. A compound according to claim 18, wherein in R₁ or R₂ the alkylsulfur is substituted with a C₆ aryl group.

32. A method of treating a disease caused by a disturbance in the activity of the androgen receptor comprising administering a compound comprising Formula I to a subject in need thereof, wherein Formula I is defined as: 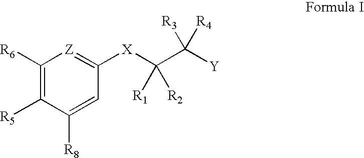
in which;
R₁ and R₂ are the same or different and independently selected from hydrogen, halogen, C₁-C₁₀ alkyl, C₁-C₁₀ substituted alkyl, C₂-C₁₀ alkenyl, C₂-C₁₀ alkynyl, C₁-C₁₀ alkoxy, C₁-C₁₀ alkenoxy, C₁-C₁₀ alkynoxy, C₁-C₁₀ alkylthio, C₁-C₁₀ alkenylthio, C₁-C₁₀ alkynylthio, C₆-C₁₀ arylthio, C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alkynylsulphoxide, C₆-C₁₀ arylsulphoxide, C₁-C₁₀ alkylarylthio, C₁-C₁₀ alkylarylsulphone, C₁-C₁₀ alkylarylsulphoxide, C₆-C₁₀ aryl, or C₅-C₂₀ heteroaryl, optionally substituted with 0, 1, 2 or 3 groups of R^{a} which groups may be the same or different; or R₁ and R₂ may together form a C₃-C₁₀ cycloalkyl group;
R₃ and R₄ are the same or different and independently selected from hydrogen, halogen, C₁-C₂₀ alkyl, C₃-C₇ cycloalkyl, C₂-C₄ alkenyl; C₂-C₄ alkynyl, C₁-C₄ alkoxy, C₁-C₄ alkenoxy, C₁-C₄ alkynoxy, C₁-C₄ alkylthio, C₁-C₄ alkenylthio, C₁-C₄ alkynylthio, C₁-C₁₀ alkylsulphone, C₁-C₁₀ alkenylsulphone, C₁-C₁₀ alkynylsulphone, C₆-C₁₀ arylsulphone, C₁-C₁₀ alkylsulphoxide, C₁-C₁₀ alkenylsulphoxide, C₁-C₁₀ alkynylsulphoxide, C₆-C₁₀ arylsulphoxide, C₁-C₁₀ alkylarylthio, C₁-C₁₀ alkylarylsulphone, C₁-C₁₀ alkylarylsulphoxide, C₆-C₁₅ aryl, C₅-C₂₀ heteroaryl optionally substituted with 0, 1, 2 or 3 groups of R^{a} which groups may be the same or different; or can together form a keto group;
R₅ is chosen from nitro, cyano, —CH₂CN, —COMe, or —SO₂CH₃;
R₆ is chosen from the group consisting of; hydrogen, C₁-C₅ alkyl, halogen, CN, CO₂H, CHF₂, CH₂F or CF₃;
R₈ is chosen from the group consisting of; hydrogen, C₁-C₅ alkyl, halogen, CHF₂, CH₂F or CF₃;
X is —NH—;
Y is chosen from hydroxy, or —NH(C₁-C₁₀ heteroaryl);
Z is chosen from CR₇ or N;
R₇ is H or C₁-C₅ alkyl;
R^{a} represents a member selected from: hydrogen, halogen, —CN, OH, CO₂H, CHO, NO₂, —NH₂, —NH(C₁-C₄); N(C₁-C₄)₂, —NH(C₆aryl), —N(C₆ aryl)₂, —NH(C₅-C₁₀ heteroaryl), and —N(C₅-C₁₀ heteroaryl)₂; or a pharmaceutically acceptable salt thereof,
with the proviso that the compound is not one of: 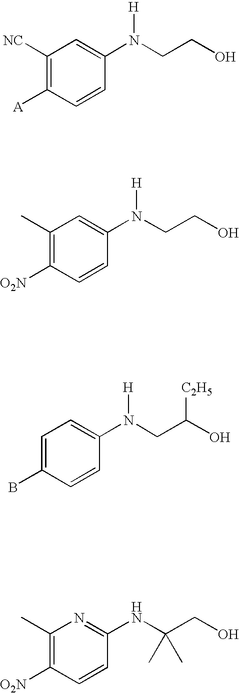
wherein A is —CN or —NO₂, and B is —CN, —NO₂ or —SO₂CH₃.

33. A method according to claim 32, wherein R₁ or/and R₂ are H, (S)-methyl, methyl, (R)-ethyl, (S)-ethyl, ethyl, (R)-propyl, (S)-propyl, propyl, (S)-butyl, S-1-methyl-propyl, S-2-methyl-propyl, (R)-isopropyl, (S)-isopropyl, isopropyl, cyclopentyl, —(CH₂)₂SMe, (R) —CH₂SCH₂Ph, (S)-benzyl, 4-chloro-benzyl, (S)-3-methyl-1-H-indole or (S)-phenyl.

34. A method according to claim 32, wherein R₃ is chosen from the group consisting of hydrogen, methyl, ethyl, phenyl, 3-hydroxy phenyl, 4-hydroxy phenyl, or forms a keto group together with R₄.

35. A method according to claim 32, wherein R₄ is H, methyl, or forms a keto group together with R₃.

36. A method according to claim 32, wherein R₅ is NO₂, CN, or CH₂CN.

37. A method according to claim 32, wherein R₆ is Me or CF₃.

38. A method according to claim 32, wherein R₇ is H or Me.

39. A method according to claim 32, wherein R₈ is H or methyl.

40. A method according to claim 32, wherein Y is —OH.

41. A method according to claim 32, wherein the compound is chosen from the group consisting of:
2-Methyl-2-(4-nitro-3-trifluoromethyl-phenylamino)-propan-1-ol;
[1-(4-Nitro-3-trifluoromethyl-phenylamino)-cyclopentyl]-methanol
(S)-2-(4-Nitro-3-trifluoromethyl-phenylamino)-3-phenyl-propan-1-ol;
(S)-2-(4-Nitro-3-trifluoromethyl-phenylamino)-butan-1-ol;
2-Methyl-2-(3-hydroxy-4-nitro-phenylamino)-propan-1-ol;
[1-(3-Methyl-4-nitro-phenylamino)-cyclopentyl]-methanol;
(S)-2-(3-Methyl-4-nitro-phenylamino)-butan-1-ol;
2-Methyl-2-(6-methyl-5-nitro-pyridine-2-ylamino)-propan-1-ol;
[1-(6-Methyl-5-nitro-pyridine-2-ylamino)-cyclopentyl]-methanol;
(S)-2-(6-Methyl-5-nitro-pyridin-2ylamino) 2-phenyl-ethanol;
(S)-2-(6-Methyl-5-nitro-pyridine-2-ylamino)-3-phenyl-propan-1-ol;
(S)-2-(6-Methyl-5-nitro-pyridin-2-ylamino)-butan-1-ol;
(DL)-3-(4-Chloro-phenyl)-2-(6-methyl-5-nitro-pyridin-2-ylamino)-propan-1-ol;
(S)-2-(6-Methyl-5-nitro-2-pyridin-2-ylamino)-propionic acid;
(S)-2-(6-Methyl-5-nitro-pyridin-2-ylamino)-propan-1-ol;
2-(2,3-Dimethyl-4-nitro-phenylamino)-2-methyl-propan-1-ol;
(S)-2-(3,5-Dimethyl-4-nitro-phenylamino)-butan-1-ol;
4-(2-Hydroxy-1,1-dimethyl-ethylamino)-2-trifluoromethyl-benzonitrile;
4-(1-Hydroxymethyl-cyclopentylamino)-2-trifluoromethyl-benzonitrile;
(S)-4-(1-Hydroxymethyl-cyclopentylamino)-2-trifluoromethyl-benzonitrile;
(R)-4-(1-Hydroxymethyl-butylamino)-2-trifluoromethyl-benzonitrile;
(S)-4-(1-Hydroxymethyl-butylamino)-2-trifluoromethyl-benzonitrile;
[4-((S)-1-Hydroxymethyl-butylamino)-2-trifluoromethyl-phenyl]-acetonitrile;
[4-((R)-1-Hydroxymethyl-butylamino)-2-trifluoromethyl-phenyl]-acetonitrile;
[4-((S)-1-Hydroxymethyl-3-methyl-butylamino)-2-trifluoromethyl-phenyl]-acetonitrile;
4-(2-Hydroxy-1,1-dimethyl-ethylamino)-2-methyl-benzonitrile;
6-(2-Hydroxy-1,1-dimethyl-ethylamino)-2-methyl-nicotinonitrile;
4-(2-Hydroxy-1,1-dimethyl-ethylamino)-2,3-dimethyl-benzonitrile;
and compounds having the formula: 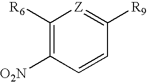
in which R₉, R₆ and Z are as defined in the following table:

| R9 | R6 | Z |
|---|---|---|
| 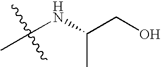 | CF₃ | CH |
| 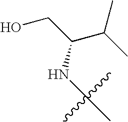 | CF₃ | CH |
| 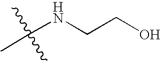 | CF₃ | CH |
| 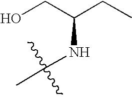 | CF₃ | CH |
| 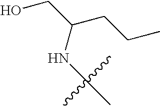 | CF₃ | CH |
| 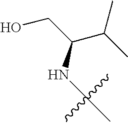 | CF₃ | CH |
| 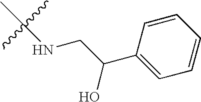 | CF₃ | CH |
| 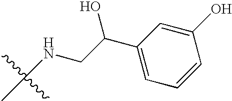 | CF₃ | CH |
| 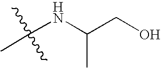 | CF₃ | CH |
| 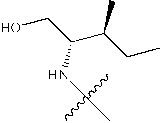 | CF₃ | CH |
| 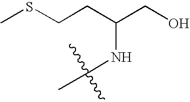 | CF₃ | CH |
| 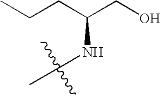 | CF₃ | CH |
| 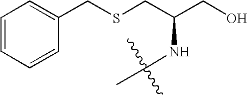 | CF₃ | CH |
| 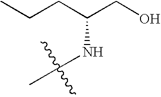 | CF₃ | CH |
| 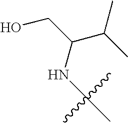 | CF₃ | CH |
| 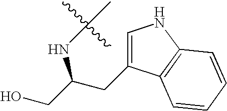 | CF₃ | CH |
| 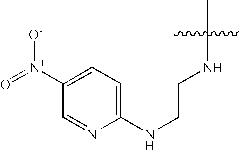 | CF₃ | CH |
| 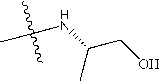 | CH₃ | N |
| 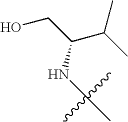 | CH₃ | N |
| 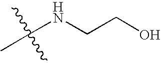 | CH₃ | N |
| 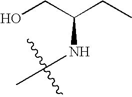 | CH₃ | N |
| 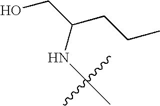 | CH₃ | N |
| 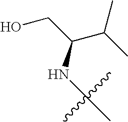 | CH₃ | N |
| 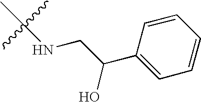 | CH₃ | N |
| 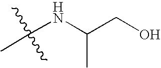 | CH₃ | N |
| 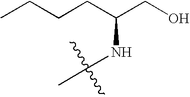 | CH₃ | N |
| 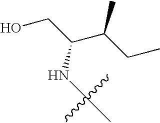 | CH₃ | N |
| 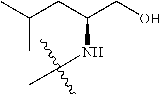 | CH₃ | N |
| 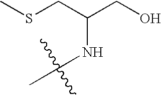 | CH₃ | N |
| 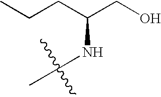 | CH₃ | N |
| 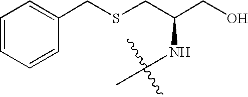 | CH₃ | N |
| 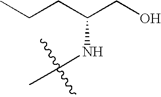 | CH₃ | N |
| 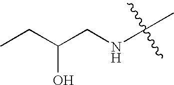 | CH₃ | N |
| 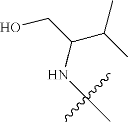 | CH₃ | N |
| 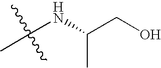 | CH₃ | CH |
| 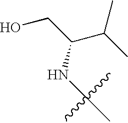 | CH₃ | CH |
| 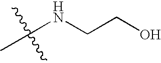 | CH₃ | CH |
| 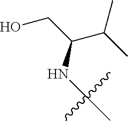 | CH₃ | CH |
| 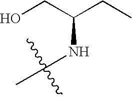 | CH₃ | CH |
| 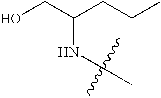 | CH₃ | CH |
| 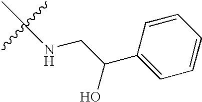 | CH₃ | CH |
| 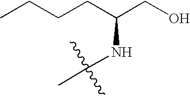 | CH₃ | CH |
| 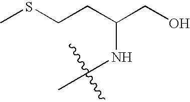 | CH₃ | CH |
| 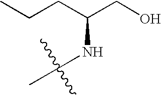 | CH₃ | CH |
| 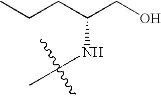 | CH₃ | CH |
| 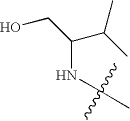 | CH₃ | CH |
| 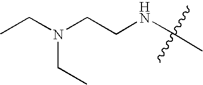 | CH₃ | CH |
| 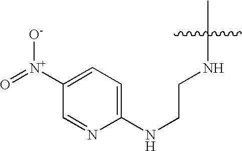 | CH₃ | CH |

2-Methyl-N-(6-methyl-5-nitro-pyridin-2-yl amino)-propan-2-ol;
4-((R)-2-Hydroxy-1-methyl-ethylamino)-2-trifluoromethyl-benzonitrile
4-((R)-1-Furan-2-ylmethyl-2-hydroxy-ethylamino)-2-trifluoromethyl-benzonitrile
(R)-3-Furan-2-yl-2-(6-methyl-5-nitro-pyridin-2-ylamino)-propan-1-ol
2-(6-Methyl-5-nitro-pyridin-2-ylamino)-heptan-1-ol
3-Cyclopentyl-2-(6-methyl-5-nitro-pyridin-2-ylamino)-propan-1-ol
[1-(4-Methanesulfonyl-3-methyl-phenylamino)-cyclopentyl]-methanol
2,2-Dimethyl-3-(6-methyl-5-nitro-pyridin-2-ylamino)-propan-1-ol
2,2-Dimethyl-3-(3-methyl-4-nitro-phenylamino)-propan-1-ol
4-((R)-1-Benzylsulfanylmethyl-2-hydroxy-ethylamino)-2-trifluoromethyl-benzonitrile
(R)-2-(6-Methyl-5-nitro-pyridin-2-ylamino)-3-phenylmethanesulfinyl-propan-1-ol
4-((R)-2-Hydroxy-1-phenylmethanesulfinylmethyl-ethylamino)-2-trifluoromethyl-benzonitrile
[1-(4-Nitro-phenylamino)-cyclopentyl]-methanol
(S)-2-(4-Nitro-phenylamino)-pentan-1-ol
[1-(2-Bromo-4-nitro-phenylamino)-cyclopentyl]-methanol
(S)-2-(2-Bromo-4-nitro-phenylamino)-pentan-1-ol
(S)-2-(2-Bromo-4-nitro-phenylamino)-4-methyl-pentan-1-ol

42. A method according to claim 32, wherein R₁ or R₂ is a C₆-C₁₀ arythio comprising an aryl-substituted sulfur-containing C₁-C₁₀ alkyl group.

43. A method according to claim 32, wherein in R₁ or R₂ the alkylsulfur is substituted with a C₆ aryl group.
